(19) United States
(12) Patent Application Publication
(10) Pub. No.: US 2022/0047710 A1
(43) Pub. Date: Feb. 17, 2022

(54) SINGLE CHAIN CONSTRUCTS

(71) Applicants: Chang Liu, St. Louis, MO (US); Daved H. Fremont, St. Louis, MO (US); Brian Edelson, Webster Groves, MO (US); Christopher A. Nelson, St. Louis, MO (US); Xiaoli Wang, St. Louis, MO (US)

(72) Inventors: Chang Liu, St. Louis, MO (US); Daved H. Fremont, St. Louis, MO (US); Brian Edelson, Webster Groves, MO (US); Christopher A. Nelson, St. Louis, MO (US); Xiaoli Wang, St. Louis, MO (US)

(73) Assignee: Washington University, St. Louis, MO (US)

(21) Appl. No.: 17/275,808

(22) PCT Filed: Sep. 12, 2019
(86) PCT No.: PCT/US2019/050837

§ 371 Date: Mar. 12, 2021

Related U.S. Application Data
(60) Provisional application No. 62/730,219, filed on Sep. 12, 2018.

(51) Int. Cl. A61K 47/68; C07K 14/725; C07K 16/30; A61P 37/06

(52) U.S. Cl. CPC A61K 47/68; C07K 14/7051; A61K 38/00; A61P 37/06; C07K 16/3092

(57) ABSTRACT

Fusion proteins are provided, the fusion proteins comprising a single-chain trimer coupled to an Fc domain of an antibody. Also provided are methods of use thereof.

### REFERENCE TO A SEQUENCE LISTING, TABLE, OR COMPUTER PROGRAM LISTING APPENDIX SUBMITTED ON A COMPACT DISC AND AN INCORPORATION-BY-REFERENCE OF THE MATERIAL ON A COMPACT DISC

The instant application contains a Sequence Listing as a text file, which is entitled “WSTL18682.WO GENE SEQUENCE LISTING” as created on Sep. 11, 2019, and is 117000 bytes in size. This sequence listing was submitted via EFS-Web in ASCII format on Sep. 12, 2019, and is hereby incorporated by reference in its entirety.

### FIELD OF THE INVENTION

The present disclosure generally relates to fusion proteins comprising a single chain construct and a fragment crystallizable (Fc) region of an antibody wherein the single chain construct comprises an antigen peptide, a β2 microglobulin, a heavy chain of a major histocompatibility complex (MHC). Also provided are methods of use thereof.

### BACKGROUND OF THE INVENTION

Donor-specific antibodies (DSA) to human leukocyte antigens (HLA) are critical barriers to life-saving organ transplantations. Existing treatment options are limited. DSA can cause hyperacute graft rejection if present at the time of transplantation. If a patient is sensitized to many different HLAs due to transfusion, pregnancy, or previous transplantation, the chance of finding a serologically compatible donor is greatly diminished. Currently, over 10,000 kidney transplant candidates in the U.S. are incompatible with ≥98% of the donor population.¹ For candidates on the waitlist who are moderate to very highly sensitized, the 5- and 8-year mortality rates are 40.8% and 56.1% respectively if no compatible donor can be identified for transplantation.² Although incompatible transplantation crossing the DSA barrier can be performed after desensitization, it has been associated with significantly worse outcomes compared to compatible kidney transplantations.³⁻⁷

DSA can also be induced post-transplantation after a memory response or de novo sensitization leading to acute and chronic antibody-mediated rejection (AMR). In the era of potent T cell immunosuppression, AMR has been recognized as a prominent risk factor for long-term graft survival.⁸⁻¹² Up to 50% of the renal graft failures in a biopsy-for-cause population have been attributed to AMR.⁹ The median 10-year renal graft survival for patients with and without de novo DSA were 57% versus 96%.¹⁰ The detrimental effect of DSA has been extensively documented for not only kidney transplantation but also hematopoietic stem cell transplantation^{13,14} and other solid organ transplantations such as lung,¹⁵⁻¹⁷ heart,¹⁸⁻²⁰ and liver transplantations.^{21,25} In summary, DSA is associated with a substantial disease burden and represents a significant threat to long-term transplant outcomes.

Existing strategies to remove DSA include plasma exchange (PEX) plus intravenous immunoglobulin (IVIG), B cell depletion by anti-CD20 (e.g., rituximab), plasma cell inhibition by proteasome inhibitors (e.g., bortezomib), and IgG endopeptidase.²⁶ Complement factors C1 and C5 inhibitors (e.g., eculizumab) have also been used to treat AMR. None has been approved by the Food and Drug Administration (FDA) for desensitization or AMR treatment. The persisting gaps between the menace of DSA and existing treatments are twofold. First, recent cohort studies and randomized trials showed limited or insignificant benefits for rituximab,²⁷⁻²⁹ bortezomib,^{30,31} eculizumab^{32,33}, or multimodality protocols including PEX and IVIG³⁴ in the management of DSA and AMR. Although IgG endopeptidase was promising for desensitization, strong memory responses post-transplant have been reported.²⁶ Second, none of the existing therapies can specifically reduce the production of DSA. Nonselective suppression of the humoral immunity has been associated with severe adverse effects including life-threatening infections.^{35,36}

Thus, there remains a need for effective methods and tools to selectively reduce production and levels of DSA in patients without suppression of overall humoral immunity.

### BRIEF SUMMARY OF THE INVENTION

A fusion protein is provided comprising a single chain trimer (SCT) and a fragment crystallizable (Fc) region of an antibody, wherein the SCT comprises an antigen peptide, a first flexible linker, a β2-microglobulin, a second flexible linker and a MHC class I heavy chain.

Another fusion protein is provided comprising a β2-microglobulin, a flexible linker, a MHC class I heavy chain and a fragment crystallizable (Fc) region of an antibody.

A dimer is provided comprising two fusion proteins as provided herein, wherein the Fc regions of the fusion proteins are covalently linked.

A fusion protein complex is provided, the complex comprising a fusion protein as provided herein and an antigen peptide, wherein the antigen peptide is complexed (e.g., affinity bound) with the fusion protein.

A nucleic acid is provided, the nucleic acid comprising a nucleotide sequence encoding a fusion protein as provided herein.

An expression vector is provided, the expression vector comprising the nucleic acid provided herein.

A host cell is provided, the host cell comprising the expression vector provided herein.

A pharmaceutical composition is provided comprising a fusion protein, fusion protein dimer, fusion protein complex or any combination thereof as described herein.

A method is provided for depleting a population of antigen-specific cells expressing a surface receptor having an affinity for a MHC-peptide complex. The method comprises contacting the cells with an effective amount of the fusion protein or fusion protein complex described herein.

A method is provided for treating antibody-mediated transplant rejection in a subject in need thereof, wherein the antibody-mediated rejection is caused by antibodies having an affinity for a foreign HLA-peptide complex. The method comprises depleting a population of B cells that express a surface receptor having an affinity for the foreign HLA-peptide complex in the subject according to the methods provided herein.

Another method is provided of treating organ transplant rejection, graft-versus-host disease, and/or blood transfusion refractoriness in a subject in need thereof. The method comprises administering to the subject a therapeutically effective amount of the fusion protein or fusion protein complex provided herein.

Another method is provided for treating antibody-mediated hemolysis in a subject in need thereof. The method comprises administering a therapeutically effective amount of the fusion protein or fusion protein complex provided herein.

Also provided is an imaging agent comprising any fusion protein described herein conjugated to a signal generating moiety.

A method for staining an antigen specific cell population is provided. The method comprises contacting the antigen specific cells with the imaging agent described herein.

Other objects and features will be in part apparent and in part pointed out hereinafter.

### BRIEF DESCRIPTION THE DRAWINGS

FIG. 1 is an illustration of an A2-Fc fusion protein (middle) compared to a standard antibody (left) and its use in selectively depleting A2-specific B cells (right).

FIG. 2.1 and FIG. 2.2 show a linearized and circular gene vector used to express an A2-Fc fusion protein.

FIG. 3 depicts fusion constructs comprising a peptide, a β2-microglobulin, a MHC heavy chain and an Fc region of an antibody. Upper panel shows the schematic of constructs (006) with a normal Fc chain. Lower panel shows the schematic of construct 007 with a mutated Fc chain that cannot initiate the complement cascade.

FIG. 4 is a diagram of a dimer formed by two fusion proteins as described herein.

FIG. 5 is a gel showing that both A2-Fc and A2-Fc_{LALAPG} form dimers that can be reduced into monomers.

FIG. 6.1, FIG. 6.2, FIG. 6.3 and FIG. 6.4 show representative scatter plots showing APC fluorescence (y-axis) relative to FITC fluorescence (x-axis) for MA2.1 cells (panels A, C, E, and G) or BB7.1 cells (panels B, D, F, and H) that were either untreated (panels A-F) or treated (panels G-H) with an anti-HLA-A2 antibody.

FIG. 7.1, FIG. 7.2, and FIG. 7.3 show representative flow cytometry plots indicating 7AAD fluorescence (y-axis) relative to FITC fluorescence (x-axis) in MA2.1 cells (left) or BB7.1 cells (right) either treated with vehicle (FIG. 7.1), A2-Fc (FIG. 7.2) or A2-Fc_{LALAPG} (FIG. 7.3).

FIG. 7.4 shows scatter plots depicting levels of BCR+ living cells after each treatment condition in FIGS. 7.1 to 7.3.

FIG. 8 shows schematics of various fusion proteins comprising alternatively, different peptides (CMVpp65, Vaccinia virus L-9mer, Dengue virus G-9mer, or Dengue virus H-9mer) or different MHC heavy chains (e.g., A*02:01 or B*07:02).

FIG. 9 shows a representative immunoblot that shows levels of fusion proteins detected in the supernatant or lysate of an expression cell line transfected with the indicated transcripts (#006, #010, #018, #019, or #020).

FIG. 10 shows a representative gel indicating the unreduced and reduced versions of fusion protein obtained in the supernatant from expressed transcripts #019, #020, and #018.

FIG. 11.1 and FIG. 11.2 depict the Luminex-based single-antigen bead (SAB) assay that can be used to detect and quantify anti-HLA antibodies (e.g., anti-A2 antibodies) in a sample. FIG. 11.1 shows the experimental flow of the test. FIG. 11.2 shows a representative plot showing the output (e.g., mean fluorescence intensity (MFI)) of an antibody sample with intensities indicative of Anti-A2 antibodies outlined.

FIG. 12 shows mean fluorescence intensity (MFI) of an antibody sample of an A11 mouse immunized with A2 cells (upper panel) or a wildtype (WT) mouse immunized with A2 cells (lower panel).

FIG. 13 indicates an experimental flow wherein A11 or B7 mice are allo-immunized to A2 donor cells and then treated in vivo with vehicle, A2-Fc, or complement resistant A2-Fc_{LALAPG}.

Corresponding reference characters indicate corresponding parts throughout the drawings.

### DETAILED DESCRIPTION OF THE INVENTION

The present disclosure provides for therapeutic compositions and related treatment methods to target donor-specific B cells or T cells for pre-transplant desensitization and for treating various transplant or graft complications (e.g., graft versus host disease, antibody mediated rejection, etc.) in the post-transplant stage. The described compositions and methods can be capable of (1) treating allosensitization and antibody mediated rejection in transplant patients; (2) treating blood transfusion refractoriness and antibody-mediated hemolysis; or (3) if expanded to autoantigens, used to treat antibody-mediated autoimmune diseases.

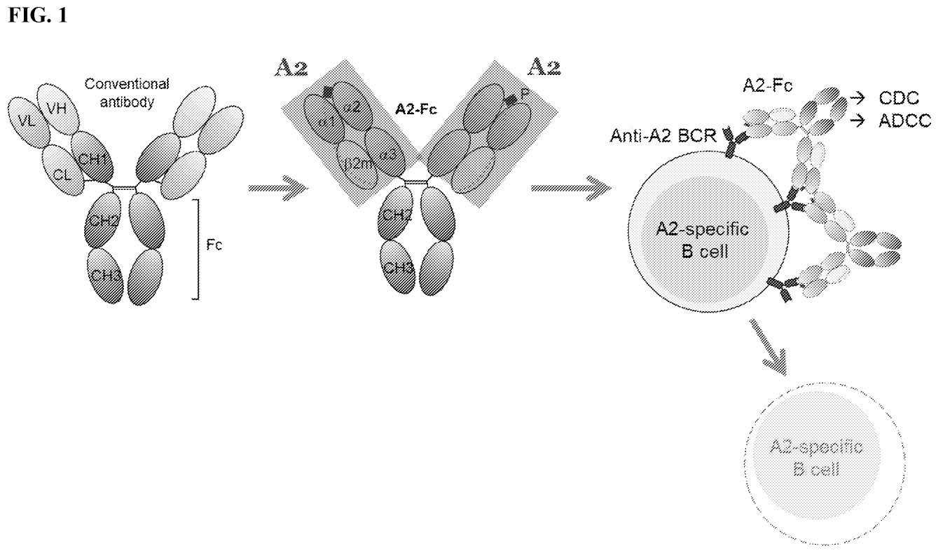

It has been discovered that fusion proteins comprising a major histocompatibility complex (MHC) specific single chain trimer (SCT) fused to a fragment crystallizable (Fc) domain of an antibody can selectively eliminate corresponding MHC specific cells (i.e., cells expressing a surface receptor having an affinity for a certain MHC-peptide complex) via complement-dependent cytotoxicity. The MHC specific single chain trimer can comprise an antigen peptide, β2-microglobulin, and a MHC heavy chain (for example, a human MHC such as HLA-A2). The present disclosure provides for a variety of MHC-Fc compositions, wherein the MHC portion comprises the single-chain trimers (SCTs). The disclosure also provides for methods of depleting an antigen specific cell population using the fusion proteins described herein. Accordingly, the fusion proteins and compositions described herein can treat antibody-mediated transplant rejections, blood transfusion refractoriness, graft versus host disease, and auto-immune diseases without inhibition of global humoral immunity (see, e.g., FIG. 1).

Various fusion proteins are described in more detail herein below.

### Fusion Proteins

### Single Chain Trimers (SCT)

The fusion proteins can comprise a single chain trimer (SCT), such as, for example, those described in U.S. Pat. Nos. 8,518,697, 8,895,020, and 8,992,937 each incorporated herein by reference in their entirety.

In various embodiments, the single chain trimer (SCT) can comprise an antigen peptide, a β2-microglobulin sequence, and a MHC class I heavy chain. Each of the components of the single chain trimer (SCT) are described below.

### Antigen Peptide

In various embodiments, the single chain trimer can comprise an antigen peptide. Preferably, the antigen peptide can bind to a corresponding MHC class I heavy chain or MHC class I-like antigen presenting molecule such as CD1 (Altamirano, M. M., et al., PNAS 98: 3288-3293, 2001). In some aspects, the antigen peptide can be that of a peptide which can be presented by a MHC class I molecule.

In general, as used herein, the term “antigen peptide” encompasses peptides derived from both “non-self” and “self” sources which can associate with the binding groove of a MHC molecule. Most preferably, the antigen peptide can associate with the binding groove of the heavy chain of the MHC molecule that is also incorporated into the SCT fusion protein.

In various aspects, the antigen peptide can be a peptide, glycopeptide, or any amino acid containing compound that is associated with a ligand binding groove of various different molecules with a MHC class I or MHC class I-like structure (Fundamental Immunology, 2d Ed., W. E Paul, ed., Ravens Press N.Y, 1989). Antigen peptides from a number of sources have been characterized in detail, including in some non-limiting examples, antigen peptides from honey bee venom allergens, dust mite allergens, toxins produced by bacteria (such as tetanus toxin) and human tissue antigens involved in autoimmune diseases. Detailed discussions of such peptides are presented in U.S. Pat. Nos. 5,595,881, 5,468,481, and 5,284,935, each incorporated herein by reference in their entirety. Other non-limiting examples of antigen peptides include those identified in the pathogenesis of rheumatoid arthritis (type II collagen), myasthenia gravis (acetyl choline receptor), and multiple sclerosis (myelin basic protein). As an additional example, suitable peptides which induce Class I MHC-restricted CTL responses against HBV antigen are disclosed in U.S. Pat. No. 6,322,789, incorporated herein by reference in its entirety.

In various configurations, an antigen peptide sequence comprised by the fusion protein described herein can comprise from about 8 to about 15 amino acid residues. For example, the antigen peptide can comprise from about 8 to about 14, from about 8 to about 13, from about 8 to about 12, from about 8 to about 11, or from about 8 to about 10 amino acid residues. As a further example, the antigen peptide can comprise about 9 amino acid residues.

In some embodiments, the antigen peptide comprises a human leukocyte antigen-A (HLA-A) restricted peptide, a HLA-B restricted peptide, a HLA-C restricted peptide, a HLA-F restricted peptide, or a HLA-G restricted peptide. For example, the antigen peptide can comprise a HLA-A restricted peptide or a HLA-B restricted peptide.

In some embodiments, the antigen peptide comprises a HLA A*02 restricted peptide, a HLA-A*11 restricted peptide, or a HLA-B*07 restricted peptide. For example, the antigen peptide can comprise a HLA-A*02:01 restricted peptide, a HLA-A*11*01 restricted peptide or a HLA-B*07:02 restricted peptide.

In some embodiments, the antigen peptide can comprise an amino acid sequence having greater than 80%, greater than 85%, or greater than 90% sequence identity to any one of SEQ ID NOs: 1-22. For example, the antigen peptide can comprise any one of SEQ ID NOs 1-22. Preferably, the antigen peptide can comprise any one of SEQ ID NOs: 1-4. For example, the antigen peptide can comprise SEQ ID NO: 1. For ease of reference, illustrative antigen peptides that may be incorporated into the fusion proteins described herein are described in Table 1. Additional peptides may be found in U.S. Pat. Nos. 8,992,937 and 8,895,020 each incorporated herein by reference in their entirety.

TABLE 1

| | | Amino Acid | SEQ ID |
|---|---|---|---|
| Name | Source | Sequence | NO: |
| CMVpp65 | Cytomegalovirus | NLVPMVATV | 1 |
| L-9mer | vaccinia virus | LPCQLMYAL | 2 |
| G-9mer | Dengue virus | GPMKLVMAF | 3 |
| H-9mer | Dengue virus | HPGFTILAL | 4 |
| EBV | Ebstein-Barr | GLCTLVAML | 5 |
| BMLF 1 | Virus | | |
| fluM1 | Influenza A | GILGFVFTL | 6 |
| | virus | | |
| G209-2M | human melanoma | IMDQVPFSV | 7 |
| G280-9V | human melanoma | YLEPGPVTV | 8 |
| OVA₂₅₇₋₂₆₄ | | SIINFEKL | 9 |
| Ova5y | | SIINYEKL | 10 |
| SIYR | | SIYRYYGL | 11 |
| VSV8 | | RGYVYQGL | 12 |
| QL9 | | QLSPFPFDL | 13 |
| MCMV pp89 | | YPHFMPTNL | 14 |
| TAX | human leukemia | LLFGYPVYV | 15 |
| NP₃₈₃₋₃₉₁ | Influenza A | SRYWAIRTR | 16 |
| MAM-A2.1 | breast cancer | LIYDSSLCDL | 17 |
| HBcAgC₁₈₋₂₇ | Hepatitis B | FLPSDFFPSV | 18 |
| HBcAgC₁₀₇₋₁₁₅ | Hepatitis B | CLTFGRETV | 19 |
| HIVgag | HIV | SLYNTVATL | 20 |
| HMeso₅₄₀₋₅₄₉ | Ovarian cancer | KLLGPHVEGL | 21 |
| HLA-C | | VMAPRTLIL | 22 |

### β2 Microglobulin

In various aspects, the fusion protein described herein can comprise a β2-microglobulin sequence. The β2 microglobulin sequence can comprise a full-length β2 microglobulin sequence as expressed on a cell surface (i.e., without a leader peptide sequence). Accordingly, in some configurations the β2-microglobulin can comprise about 99 amino acids. In various aspects, the β2-microglobulin can comprise a human β2-microglobulin or a murine β2-microglobulin. In some embodiments, the β2 microglobulin can comprise an amino acid sequence having greater than 80%, greater than 85%, greater than 90%, or greater than 95% sequence identity to SEQ ID NO: 23 or 24. For example, the β2 microglobulin can comprise a β2 microglobulin having an amino acid sequence comprising SEQ ID NO: 23 or 24. In some embodiments, the β2-microglobulin comprises a human β2 microglobulin having an amino acid sequence comprising SEQ ID NO: 24.

TABLE 2

| β2 micro- | | SEQ ID |
|---|---|---|
| globulin | Sequence | NO: |
| murine | IQKTPQIQVYSRHPPENGKPNINCYV | 23 |
| | TQFHPPHIEIQMLKNGKKIPKVEMSD | |
| | MSFSKDWSFYILAHTEFTPTETDTYA | |
| | CRVKHASMAEPKTVYWDRDM | |
| human | IQRTPKIQVYSRHPAENGKSNFLNCY | 24 |
| | VSGFHPSDIEVDLLKNGERIEKVEHS | |
| | DLSFSKDWSFYLLYYTEFTPTEKDEY | |
| | ACRVNHVTLSQPKIVKWDRDM | |

### MHC Class I Heavy Chain

In various embodiments, the fusion proteins described herein can comprise a MHC class I heavy chain. The MHC class I heavy chain can be a human MHC class I heavy chain or a murine MHC class I heavy chain. The murine MHC class I heavy chain can comprise a murine MHC-K, MHC-D or MHC-L class 1 heavy chain. The human MHC class I heavy chain can comprise a human leukocyte antigen (HLA) heavy chain. For example, the MHC class I heavy chain can comprise a HLA-A, HLA-B, HLA-C, HLA-E, HLA-F, or a HLA-G heavy chain. For example, the MHC class I heavy chain can comprise a HLA-A or a HLA-B heavy chain. Further, the HLA-A heavy chain can comprise a HLA-A*02 (e.g., a HLA-A*02:01) or a HLA-A*11 (e.g., a HLA-A*11:01) heavy chain. Alternatively, the HLA-B heavy chain can comprise a HLA-B*07 (e.g., a HLA-B*07:02) heavy chain.

In some configurations, the MHC class I heavy chain sequence can include single amino acid substitutions, additions and/or deletions, such as a substitution of the residue at position 84, (e.g., a tyrosine), position 80 (e.g., a threonine) or position 86 (e.g., an alanine) with a non-aromatic amino acid other than proline. In these configurations, the amino acid substitution can comprise a standard amino acid such as leucine (L), isoleucine (I), valine (V), serine (S), threonine (T), alanine (A), histidine (H), glutamine (Q), asparagine (N), lysine (K), aspartic acid (D), glutamic acid (E), cysteine (C), arginine (R), glycine or can be a modified or unusual amino acid such as an amino acid recited in WIPO standard ST.25 (1998), Appendix 2, Table 4, which is incorporated by reference herein. As explained in more detail below, the amino acid substitution at position 84, position 80 or position 86 can comprise a cysteine. In some embodiments, the amino acid substitution at position 84 comprises an alanine.

In various embodiments, the MHC class I heavy chain can comprise any one of SEQ ID NOs: 25-30, as shown in Table 3, below. In various embodiments, the MHC class I heavy chain comprises an amino acid sequence having at least 90%, at least 95%, or at least 99% sequence identity to any one of SEQ ID NOs: 26, 28, and 30, comprising at least one amino acid substitution selected from Y84A, Y84C, T80C, and A86C. For ease of reference, Table 3 provides native MHC sequences (e.g., HLA-A*02:01, HLA-A*11:01, and HLA-B*07:02, SEQ ID NOs: 26, 28, and 30) as well as MHC sequences comprising a Y84C substitution (SEQ ID NOs: 25, 27, and 29). The cysteine (C) or native tyrosine (Y) at position 84 in each sequence is bolded and underlined.

TABLE 3

| MHC Class I | | SEQ ID |
|---|---|---|
| Heavy Chain | Sequence | NO: |
| HLA-A*02:01 | GSHSMRYFFTSVSRPGRGEPRFIAVGYVDDTQFVRFDS | 25 |
| mutated_Y84C | DAASQRMEPRAPWIEQEGPEYWDGETRKVKAHSQTHRV | |
| | DLGTLRGCYNQSEAGSHTVQRMYGCDVGSDWRFLRGYH | |
| | QYAYDGKDYIALKEDLRSWTAADMAAQTTKHKWEAAHV | |
| | AEQLRAYLEGTCVEWLRRYLENGKETLQRTDAPKTHMT | |
| | HHAVSDHEATLRCWALSFYPAEITLTWQRDGEDQTQDT | |
| | ELVETRPAGDGTFQKWAAVVVPSGQEQRYTCHVQHEGL | |
| | PKPLTLRWEPSSQPT | |
| HLA-A*02:01 | GSHSMRYFFTSVSRPGRGEPRFIAVGYVDDTQFVRFDS | 26 |
| | DAASQRMEPRAPWIEQEGPEYWDGETRKVKAHSQTHRV | |
| | DLGTLRGYYNQSEAGSHTVQRMYGCDVGSDWRFLRGYH | |
| | QYAYDGKDYIALKEDLRSWTAADMAAQTTKHKWEAAHV | |
| | AEQLRAYLEGTCVEWLRRYLENGKETLQRTDAPKTHMT | |
| | HHAVSDHEATLRCWALSFYPAEITLTWQRDGEDQTQDT | |
| | ELVETRPAGDGTFQKWAAVVVPSGQEQRYTCHVQHEGL | |
| | PKPLTLRWEPSSQPT | |
| HLA-A*11:01 | GSHSMRYFYTSVSRPGRGEPRFIAVGYVDDTQFVRFDS | 27 |
| mutated_Y84C | DAASQRMEPRAPWIEQEGPEYWDQETRNVKAQSQTDRV | |
| | DLGTLRGCYNQSEDGSHTIQIMYGCDVGPDGRFLRGYR | |
| | QDAYDGKDYIALNEDLRSWTAADMAAQITKRKWEAAHA | |
| | AEQQRAYLEGRCVEWLRRYLENGKETLQRTDPPKTHMT | |
| | HHPISDHEATLRCWALGFYPAEITLTWQRDGEDQTQDT | |
| | ELVETRPAGDGTFQKWAAVVVPSGEEQRYTCHVQHEGL | |
| | PKPLTLRWELSSQPT | |
| HLA-A*11:01 | GSHSMRYFYTSVSRPGRGEPRFIAVGYVDDTQFVRFDS | 28 |
| | DAASQRMEPRAPWIEQEGPEYWDQETRNVKAQSQTDRV | |
| | DLGTLRGYYNQSEDGSHTIQIMYGCDVGPDGRFLRGYR | |
| | QDAYDGKDYIALNEDLRSWTAADMAAQITKRKWEAAHA | |
| | AEQQRAYLEGRCVEWLRRYLENGKETLQRTDPPKTHMT | |
| | HHPISDHEATLRCWALGFYPAEITLTWQRDGEDQTQDT | |
| | ELVETRPAGDGTFQKWAAVVVPSGEEQRYTCHVQHEGL | |
| | PKPLTLRWELSSQPT | |
| HLA-B*07:02 | GSHSMRYFYTSVSRPGRGEPRFISVGYVDDTQFVRFDS | 29 |
| mutated_Y84C | DAASPREEPRAPWIEQEGPEYWDRNTQIYKAQAQTDRE | |
| | SLRNLRGCYNQSEAGSHTLQSMYGCDVGPDGRLLRGHD | |
| | QYAYDGKDYIALNEDLRSWTAADTAAQITQRKWEAARE | |
| | AEQRRAYLEGECVEWLRRYLENGKDKLERADPPKTHVT | |
| | HHPISDHEATLRCWALGFYPAEITLTWQRDGEDQTQDT | |
| | ELVETRPAGDRTFQKWAAVVVPSGEEQRYTCHVQHEGL | |
| | PKPLTLRWEPSSQST | |
| HLA-B*07:02 | GSHSMRYFYTSVSRPGRGEPRFISVGYVDDTQFVRFDS | 30 |
| | DAASPREEPRAPWIEQEGPEYWDRNTQIYKAQAQTDRE | |
| | SLRNLRGYYNQSEAGSHTLQSMYGCDVGPDGRLLRGHD | |
| | QYAYDGKDYIALNEDLRSWTAADTAAQITQRKWEAARE | |
| | AEQRRAYLEGECVEWLRRYLENGKDKLERADPPKTHVT | |
| | HHPISDHEATLRCWALGFYPAEITLTWQRDGEDQTQDT | |
| | ELVETRPAGDRTFQKWAAVVVPSGEEQRYTCHVQHEGL | |
| | PKPLTLRWEPSSQST | |

### Flexible Linkers

In various aspects, the SCT can further comprise linker sequences, that is, sequences which impart flexibility between neighboring domains. In some aspects, the first flexible linker can extend between the antigen peptide and the β2-microglobulin. In some aspects, the first flexible linker sequence can comprise at least 8, at least 10, at least 11, at least 12, at least 13, at least 14, at least 15, at least 16, at least 17, at least 18, at least 19 or at least 20 amino acid residues. In some aspects, a second flexible linker can extend between the β2 microglobulin and the MHC class I heavy chain sequence and can comprise, in some configurations, at least 8, at least 10, at least 11, at least 12, at least 13, at least 14, at least 15, at least 16, at least 17, at least 18, at least 19 or at least 20 amino acid residues. In various embodiments, the first and second flexible linkers can each independently comprises from 10 to 25 amino acid residues, or from 15 to 20 amino acid residues.

In various embodiments, the first and second flexible linkers can each independently contain at least about 80% glycine (G), alanine (A) and/or serine (S) residues. In further embodiments, the first and second flexible linkers can each independently comprises at least about 80% glycine residues. Illustrative linker sequences and their SEQ ID NOs are provided in Table 4 below.

TABLE 4

| | | First | |
|---|---|---|---|
| | | (1st)/ | |
| | | Second | SEQ ID |
| Group | Sequence | (2nd) | NO: |
| Linkers not | GGGASGGGGS | 1st | 31 |
| comprising | | | |
| a cysteine | | | |
| | GGGGSGGGGS | 1st | 32 |
| | GGGASGGGGSGGGGS | 1st | 33 |
| | GGGGSGGGGSGGGGS | 1st | 34 |
| | GGGGSGGGGSGGGGSGGGGS | 2nd | 35 |
| | GGGSGGGSGGGSGGGSGGGS | 2nd | 36 |
| Linkers | CGGASGGGGSGGGGS | 1st | 37 |
| comprising | | | |
| a cysteine | | | |
| | GCGASGGGGSGGGGS | 1st | 38 |
| | GGCASGGGGSGGGGS | 1st | 39 |
| | GGGCSGGGGSGGGGS | 1st | 40 |
| | GGGACGGGGSGGGGS | 1st | 41 |

In various configurations, the first and second linkers can have an amino acid sequence comprising any one of SEQ ID NOs: 31-41. In various embodiments, the first linker can have an amino acid sequence comprising any one of SEQ ID NOs: 31-34 and 37-41. In some embodiments, as described in more detail below, the first linker can comprise a cysteine residue. Accordingly, the first linker can have an amino acid sequence comprising any one of SEQ ID NOs: 37-41. For example the first linker can have an amino acid sequence comprising SEQ ID NO: 38. In various embodiments, the second linker can have an amino acid sequence comprising SEQ ID NO: 35 or SEQ ID NO: 36. For example, the second linker can have an amino acid sequence comprising SEQ ID NO: 35.

### Covalent Linkage and Structure of SCT Domain of Fusion Protein

In various embodiments, a first residue in the first flexible linker and a second residue in the MHC class I heavy chain are linked by a covalent bond. In some embodiments, the first residue is a first cysteine residue and the second residue is a second cysteine residue and the covalent bond comprises a disulfide bridge. When the covalent bond comprises a disulfide bridge, the resulting linkage is also called a disulfide trap. Disulfide traps are described in U.S. Pat. No. 8,992,937 which is incorporated herein by reference in its entirety. The disulfide trap locks the antigen peptide into the MHC class I binding groove and so it is particularly suitable for fusion proteins comprising an antigen peptide having a lower affinity for the MHC class I domain.

Accordingly, the fusion proteins described herein can comprise a first flexible linker comprising the first cysteine residue. Preferably, the cysteine residue occurs within the first five amino acid residues of the first flexible linker extending from the C-terminus of the antigen peptide. For example, the cysteine residue can be the first, the second or the third amino acid residue of the first flexible linker (that is, as counted from the C-terminus of the antigen peptide). For instance, the cysteine residue can be the second amino acid residue of the first flexible linker. As described above, suitable first linker sequences that may be used to form the disulfide trap and that may be incorporated into the fusion proteins described herein can comprise any one of SEQ ID NOs: 37-41 (e.g., SEQ ID NO: 38).

As described above, the second cysteine residue preferably is located in the MHC class I domain of the fusion protein. In various configurations, the second cysteine can be a mutation in a native MHC class I heavy chain. For example, the mutation can be a cysteine which substitutes for an amino acid of the MHC class I heavy chain, or a cysteine addition to the MHC class I heavy chain. In various embodiments, the second cysteine residue can be located from about 1 to about 100, from about 10 to about 100, from about 20 to about 100, from about 30 to about 100, from about 40 to about 100, from about 50 to about 100, from about 55 to about 100, from about 60 to about 100, from about 60 to about 90, from about 65 to about 90, from about 70 to about 90, or from about 80 to about 90 amino acid residues from the amino terminus of the MHC class I heavy chain. Preferably, the second cysteine residue is located 80, 84 or 86 amino acid residues from the amino terminus of the MHC class I heavy chain. In some embodiments, the second cysteine residue can be a Y84C substitution (i.e., a substitution of tyrosine-84 of a MHC class I heavy chain with a cysteine). In other embodiments, the second cysteine can be a T80C substitution (i.e., a substitution of threonine-80 with a cysteine). In further embodiments, the second cysteine can be a A86C substitution (i.e., a substitution of an alanine 86 with a cysteine).

Accordingly, in various embodiments, the fusion protein described herein can comprise a MHC class I heavy chain having at least 90%, at least 95%, or at least 99% sequence identity to any one of SEQ ID NOs: 26, 28, and 30, and further comprising at least one amino acid substitution selected from the group consisting of Y84C, T80C, and A86C. For example, the fusion protein can comprise a MHC class I heavy chain comprising any one of SEQ ID NOs: 25, 27, and 29. In various embodiments, the MHC class I heavy chain comprises SEQ ID NO: 25.

The SCT portion of the fusion protein consequently comprises from amino to carboxy order: an antigen peptide, a first flexible linker (preferably further comprising a first cysteine residue), a β2-microglobulin, a second flexible linker, and a MHC class I heavy chain (preferably containing at least one substitution or addition such that it also comprises a second cysteine residue). When synthesized, the SCT portion of the fusion protein achieves a quaternary structure that replicates the conformation of a native MHC class I protein displaying an antigen on a cell.

### Fragment Crystallizable (Fc) Region

As described above, the fusion protein further comprises a fragment crystallizable region of an antibody. As is understood in the art, the Fc portion of an antibody comprises the relatively constant portion (and can also be called the “constant region” of the antibody) which interacts with receptors on immune cells and can initiate the complement system. The Fc region can be from a mammalian antibody (e.g., obtained from human, mouse, rabbit or goat). In some embodiments, the Fc region comprises an Fc region of a human or murine antibody. Other Fc regions are known in the art.

The Fc region can be obtained from an IgG, an IgA, an IgD, an IgM or an IgE antibody. Preferably, the fusion proteins described herein comprise an IgG Fc domain. For example, the fusion protein can comprise a murine mIgG2-Fc domain. In various embodiments, the Fc domain is located downstream of the SCT portion of the fusion protein. That is, the Fc domain is fused to the carboxy terminus of the MHC type I heavy chain. In various embodiments, the Fc domain is fused indirectly to the carboxy terminus of the MHC type I heavy chain (that is, through a short linker of one or two amino acids). In various embodiments, the short linker may be translated from a restriction enzyme site (for example, BgIII) that is included in the nucleic acid transcript encoding the full-length fusion protein (discussed below). For example, the BgIII restriction site (AGATCT) encodes two amino acids (R-S) which may be found at the amino terminus to the Fc domain in the full-fusion protein. The BgIII restriction site is illustrative. A skilled artisan will appreciate many other restriction enzyme sites that may be incorporated into the final protein at or near this location.

As noted above, many Fc portions of antibodies can initiate the complement system, thus triggering the degradation of the antibody target. Accordingly, in various embodiments, the Fc regions of the fusion proteins described herein may also initiate complement. For ease of reference, an illustrative Fc region that may be incorporated into the fusion proteins described herein is provided as SEQ ID NO: 42 in Table 5 below. Also provided is a mutated version of the Fc domain (SEQ ID NO: 43) having three amino acid substitutions (L19A, L20A and P113G) relative to SEQ ID NO: 42. However, these amino acid substitutions (bolded and underlined in Table 5) render the Fc domain incapable of initiating complement.

TABLE 5
Fc Region

| | | SEQ |
|---|---|---|
| | | ID |
| Fc Region | Sequence | NO: |
| mIgG2-Fc | PRGPTIKPCPPCKCPAPNLLGGPSVFI | 42 |
| | FPPKIKDVLMISLSPIVTCVVVDVSED | |
| | DPDVQISWFVNNVEVHTAQTQTHREDY | |
| | NSTLRVVSALPIQHQDWMSGKEFKCKV | |
| | NNKDLPAPIERTISKPKGSVRAPQVYV | |
| | LPPPEEEMTKKQVTLTCMVTDFMPEDI | |
| | YVEWTNNGKTELNYKNTEPVLDSDGSY | |
| | FMYSKLRVEKKNWVERNSYSCSVVHEG | |
| | LHNHHTTKSFSRTPGK | |
| mIgG2-Fc | PRGPTIKPCPPCKCPAPNAAGGPSVFI | 43 |
| mutated_L19A/ | FPPKIKDVLMISLSPIVTCVVVDVSED | |
| L20A/P113G | DPDVQISWFVNNVEVHTAQTQTHREDY | |
| | NSTLRVVSALPIQHQDWMSGKEFKCKV | |
| | NNKDLGAPIERTISKPKGSVRAPQVYV | |
| | LPPPEEEMTKKQVTLTCMVTDFMPEDI | |
| | YVEWTNNGKTELNYKNTEPVLDSDGSY | |
| | FMYSKLRVEKKNWVERNSYSCSVVHEG | |
| | LHNHHTTKSFSRTPGK | |

In various embodiments, the Fc region can comprise an amino acid sequence having at least 80% at least 85%, at least 90%, at least 95%, or at least 99% sequence identity to SEQ ID NO: 42. In other embodiments, the Fc region can comprise an amino acid sequence comprising SEQ ID NO: 42.

### Leader Peptide

In various embodiments, the fusion protein may further comprise a leader peptide. This peptide can be located at the amino terminus of the fusion protein and facilitate the expression and secretion of the fusion protein in an expression system (as described in more detail below). In certain embodiments, the leader peptide may be cleaved by the host cell.

An illustrative leader peptide that may be incorporated into the fusion proteins described herein is provided in Table 6 below.

TABLE 6

| Leader Peptide Amino | SEQ ID |
|---|---|
| Acid Sequence | NO: |
| MARSVTLVFLVLVSLTGLYA | 44 |

### Structure of Complete Fusion Protein

In accordance with the disclosure above, the fusion proteins described herein can comprise an antigen peptide, a first flexible linker, a β2-microglobulin, a second flexible linker, a MHC type I heavy chain, and an Fc region of an antibody. Preferably, the fusion proteins comprise in amino to carboxy terminal order: an antigen peptide, a first flexible linker, a β2-microglobulin, a second flexible linker, a MHC type I heavy chain, and an Fc region of an antibody. Optionally, the fusion protein may further comprise a leader peptide connected at its C-terminus to the amino terminus of the antigen peptide. For example, the fusion protein may further comprise a leader peptide having SEQ ID NO: 44 at its amino terminus. This leader peptide may be transiently present during expression and cleaved during maturation.

Illustrative fusion proteins with (SEQ ID NOs: 45-50) and without (SEQ ID NOs: 51-56) leader peptides are provided in Table 7 below. The leader peptide in SEQ ID NOs: 45-50 is bolded and underlined. The amino acid residues (R-S) encoded by a BgIII restriction site discussed above included in the fusion protein are italicized, bolded and underlined in all sequences.

TABLE 7

| | Amino Acid Sequence | SEQ | Amino Acid Sequence | SEQ |
|---|---|---|---|---|
| Fusion | (with leader | ID | (without leader | ID |
| Protein | peptide) | NO: | peptide) | NO: |
| #006 | MARSVTLVFLVLVSLTGLYA | 45 | NLVPMVATVGCGASGGGGSG | 51 |
| (CMVpp65- | NLVPMVATVGCGASGGGGSG | | GGGSIQRTPKIQVYSRHPAE | |
| A2-Fc) | GGGSIQRTPKIQVYSRHPAE | | NGKSNFLNCYVSGFHPSDIE | |
| | NGKSNFLNCYVSGFHPSDIE | | VDLLKNGERIEKVEHSDLSF | |
| | VDLLKNGER1EKVEHSDLSF | | SKDWSFYLLYYTEFTPIEKD | |
| | SKDWSFYLLYYTEFTPIEKD | | EYACRVNHVTLSQPKIVKWD | |
| | EYACRVNHVTLSQPKIVKWD | | RDMGGGGSGGGGSGGGGSGG | |
| | RDMGGGGSGGGGSGGGGSGG | | GGSGSHSMRYFFTSVSRPGR | |
| | GGSGSHSMRYFFTSVSRPGR | | GEPRFIAVGYVDDTQFVRFD | |
| | GEPRFIAVGYVDDTQFVRFD | | SDAASQRMEPRAPWIEQEGP | |
| | SDAASQRMEPRAPWIEQEGP | | EYWDGETRKVKAHSQTHRVD | |
| | EYWDGETRKVKAHSQTHRVD | | LGTLRGCYNQSEAGSHTVQR | |
| | LGTLRGCYNQSEAGSHTVQR | | MYGCDVGSDWRFLRGYHQYA | |
| | MYGCDVGSDWRFLRGYHQYA | | YDGKDYIALKEDLRSWTAAD | |
| | YDGKDYIALKEDLRSWTAAD | | MAAQTTKHKWEAAHVAEQLR | |
| | MAAQTTKHKWEAAHVAEQLR | | AYLEGTCVEWLRRYLENGKE | |
| | AYLEGTCVEWLRRYLENGKE | | TLQRTDAPKTHMTHHAVSDH | |
| | TLQRTDAPKTHMTHHAVSDH | | EATLRCWALSFYPAEITLTW | |
| | EATLRCWALSFYPAEITLTW | | QRDGEDQTQDTELVETRPAG | |
| | QRDGEDQTQDTELVETRPAG | | DGTFQKWAAVVVPSGQEQRY | |
| | DGTFQKWAAVVVPSGQEQRY | | TCHVQHEGLPKPLTLRWEPS | |
| | TCHVQHEGLPKPLTLRWEPS | | SQPT [symbol] PRGPTIKPCPPCKC | |
| | SQPT [symbol] PRGPTIKPCPPCKC | | PAPNLLGGPSVFIFPPKIKD | |
| | PAPNLLGGPSVFIFPPKIKD | | VLMISLSPIVTCVVVDVSED | |
| | VLMISLSPIVTCVVVDVSED | | DPDVQISWFVNNVEVHTAQT | |
| | DPDVQISWFVNNVEVHTAQT | | QTHREDYNSTLRVVSALPIQ | |
| | QTHREDYNSTLRVVSALPIQ | | HQDWMSGKEFKCKVNNKDLP | |
| | HQDWMSGKEFKCKVNNKDLP | | APIERTISKPKGSVRAPQVY | |
| | APIERTISKPKGSVRAPQVY | | VLPPPEEEMTKKQVTLTCMV | |
| | VLPPPEEEMTKKQVTLTCMV | | TDFMPEDIYVEWTNNGKTEL | |
| | TDFMPEDIYVEWTNNGKTEL | | NYKNTEPVLDSDGSYFMYSK | |
| | NYKNTEPVLDSDGSYFMYSK | | LRVEKKNWVERNSYSCSVVH | |
| | LRVEKKNWVERNSYSCSVVH | | EGLHNHHTTKSFSRTPGK | |
| | EGLHNHHTTKSFSRTPGK | | | |
| #007 | MARSVTLVFLVLVSLTGLYA | 46 | NLVPMVATVGCGASGGGGSG | 52 |
| (CMVpp65- | NLVPMVATVGCGASGGGGSG | | GGGSIQRTPKIQVYSRHPAE | |
| A2- | GGGSIQRTPKIQVYSRHPAE | | NGKSNFLNCYVSGFHPSDIE | |
| Fc_{LALAPG}) | NGKSNFLNCYVSGFHPSDIE | | VDLLKNGERIEKVEHSDLSF | |
| | VDLLKNGERIEKVEHSDLSF | | SKDWSFYLLYYTEFTPIEKD | |
| | SKDWSFYLLYYTEFTPIEKD | | EYACRVNHVTLSQPKIVKWD | |
| | EYACRVNHVTLSQPKIVKWD | | RDMGGGGSGGGGSGGGGSGG | |
| | RDMGGGGSGGGGSGGGGSGG | | GGSGSHSMRYFFTSVSRPGR | |
| | GGSGSHSMRYFFTSVSRPGR | | GEPRFIAVGYVDDTQFVRFD | |
| | GEPRFIAVGYVDDTQFVRFD | | SDAASQRMEPRAPWIEQEGP | |
| | SDAASQRMEPRAPWIEQEGP | | EYWDGETRKVKAHSQTHRVD | |
| | EYWDGETRKVKAHSQTHRVD | | LGTLRGCYNQSEAGSHTVQR | |
| | LGTLRGCYNQSEAGSHTVQR | | MYGCDVGSDWRFLRGYHQYA | |
| | MYGCDVGSDWRFLRGYHQYA | | YDGKDYIALKEDLRSWTAAD | |
| | YDGKDYIALKEDLRSWTAAD | | MAAQTTKHKWEAAHVAEQLR | |
| | MAAQTTKHKWEAAHVAEQLR | | AYLEGTCVEWLRRYLENGKE | |
| | AYLEGTCVEWLRRYLENGKE | | TLQRTDAPKTHMTHHAVSDH | |
| | TLQRTDAPKTHMTHHAVSDH | | EATLRCWALSFYPAEITLTW | |
| | EATLRCWALSFYPAEITLTW | | QRDGEDQTQDTELVETRPAG | |
| | QRDGEDQTQDTELVETRPAG | | DGTFQKWAAVVVPSGQEQRY | |
| | DGTFQKWAAVVVPSGQEQRY | | TCHVQHEGLPKPLTLRWEPS | |
| | TCHVQHEGLPKPLTLRWEPS | | SQPT [symbol] PRGPTIKPCPPCKC | |
| | SQPT [symbol] PRGPTIKPCPPCKC | | PAPNAAGGPSVFIFPPKIKD | |
| | PAPNAAGGPSVFIFPPKIKD | | VLMISLSPIVTCVVVDVSED | |
| | VLMISLSPIVTCVVVDVSED | | DPDVQISWFVNNVEVHTAQT | |
| | DPDVQISWFVNNVEVHTAQT | | QTHREDYNSTLRVVSALPIQ | |
| | QTHREDYNSTLRVVSALPIQ | | HQDWMSGKEFKCKVNNKDLG | |
| | HQDWMSGKEFKCKVNNKDLG | | APIERTISKPKGSVRAPQVY | |
| | APIERTISKPKGSVRAPQVY | | VLPPPEEEMTKKQVTLTCMV | |
| | VLPPPEEEMTKKQVTLTCMV | | TDFMPEDIYVEWTNNGKTEL | |
| | TDFMPEDIYVEWTNNGKTEL | | NYKNTEPVLDSDGSYFMYSK | |
| | NYKNTEPVLDSDGSYFMYSK | | LRVEKKNWVERNSYSCSVVH | |
| | LRVEKKNWVERNSYSCSVVH | | EGLHNHHTTKSFSRTPGK | |
| | EGLHNHHTTKSFSRTPGK | | | |
| #010 | MARSVTLVFLVLVSLTGLYA | 47 | NLVPMVATVGCGASGGGGSG | 53 |
| (CMVpp65- | NLVPMVATVGCGASGGGGSG | | GGGSIQRTPKIQVYSRHPAE | |
| B7-Fc) | GGGSIQRTPKIQVYSRHPAE | | NGKSNFLNCYVSGFHPSDIE | |
| | NGKSNFLNCYVSGFHPSDIE | | VDLLKNGERIEKVEHSDLSF | |
| | VDLLKNGER1EKVEHSDLSF | | SKDWSFYLLYYTEFTP1EKD | |
| | SKDWSFYLLYYTEFTP1EKD | | EYACRVNHVTLSQPKIVKWD | |
| | EYACRVNHVTLSQPKIVKWD | | RDMGGGGSGGGGSGGGGSGG | |
| | RDMGGGGSGGGGSGGGGSGG | | GGSGSHSMRYFYTSVSRPGR | |
| | GGSGSHSMRYFYTSVSRPGR | | GEPRFISVGYVDDTQFVRFD | |
| | GEPRFISVGYVDDTQFVRFD | | SDAASPREEPRAPWIEQEGP | |
| | SDAASPREEPRAPW1EQEGP | | EYWDRNTQIYKAQAQTDRES | |
| | EYWDRNTQIYKAQAQTDRES | | LRNLRGCYNQSEAGSHTLQS | |
| | LRNLRGCYNQSEAGSHTLQS | | MYGCDVGPDGRLLRGHDQYA | |
| | MYGCDVGPDGRLLRGHDQYA | | YDGKDYIALNEDLRSWTAAD | |
| | YDGKDYIALNEDLRSWTAAD | | TAAQITQRKWEAAREAEQRR | |
| | TAAQITQRKWEAAREAEQRR | | AYLEGECVEWLRRYLENGKD | |
| | AYLEGECVEWLRRYLENGKD | | KLERADPPKTHVTHHPISDH | |
| | KLERADPPKTHVTHHPISDH | | EATLRCWALGFYPAEITLTW | |
| | EATLRCWALGFYPAEITLTW | | QRDGEDQTQDTELVETRPAG | |
| | QRDGEDQTQDTELVETRPAG | | DRTFQKWAAVVVPSGEEQRY | |
| | DRTFQKWAAVVVPSGEEQRY | | TCHVQHEGLPKPLTLRWEPS | |
| | TCHVQHEGLPKPLTLRWEPS | | SQST [symbol] PRGPTIKPCPPCKC | |
| | SQST [symbol] PRGPTIKPCPPCKC | | PAPNLLGGPSVFIFPPKIKD | |
| | PAPNLLGGPSVFIFPPKIKD | | VLMISLSPIVTCVVVDVSED | |
| | VLMISLSPIVTCVVVDVSED | | DPDVQISWFVNNVEVHTAQT | |
| | DPDVQISWFVNNVEVHTAQT | | QTHREDYNSTLRVVSALPIQ | |
| | QTHREDYNSTLRVVSALPIQ | | HQDWMSGKEFKCKVNNKDLP | |
| | HQDWMSGKEFKCKVNNKDLP | | APIERTISKPKGSVRAPQVY | |
| | APIERTISKPKGSVRAPQVY | | VLPPPEEEMTKKQVTLTCMV | |
| | VLPPPEEEMTKKQVTLTCMV | | TDFMPEDIYVEWTNNGKTEL | |
| | TDFMPEDIYVEWTNNGKTEL | | NYKNTEPVLDSDGSYFMYSK | |
| | NYKNTEPVLDSDGSYFMYSK | | LRVEKKNWVERNSYSCSVVH | |
| | LRVEKKNWVERNSYSCSVVH | | EGLHNHHTTKSFSRTPGK | |
| | EGLHNHHTTKSFSRTPGK | | | |
| #018 | MARSVTLVFLVLVSLTGLYA | 48 | LPCQLMYALGCGASGGGGSG | 54 |
| (VVL9mer- | LPCQLMYALGCGASGGGGSG | | GGGSIQRTPKIQVYSRHPAE | |
| B7-Fc) | GGGSIQRTPKIQVYSRHPAE | | NGKSNFLNCYVSGFHPSDIE | |
| | NGKSNFLNCYVSGFHPSDIE | | VDLLKNGERIEKVEHSDLSF | |
| | VDLLKNGERIEKVEHSDLSF | | SKDWSFYLLYYTEFTP1EKD | |
| | SKDWSFYLLYYTEFTP1EKD | | EYACRVNHVTLSQPKIVKWD | |
| | EYACRVNHVTLSQPKIVKWD | | RDMGGGGSGGGGSGGGGSGG | |
| | RDMGGGGSGGGGSGGGGSGG | | GGSGSHSMRYFYTSVSRPGR | |
| | GGSGSHSMRYFYTSVSRPGR | | GEPRFISVGYVDDTQFVRFD | |
| | GEPRFISVGYVDDTQFVRFD | | SDAASPREEPRAPWIEQEGP | |
| | SDAASPREEPRAPW1EQEGP | | EYWDRNTQIYKAQAQTDRES | |
| | EYWDRNTQIYKAQAQTDRES | | LRNLRGCYNQSEAGSHTLQS | |
| | LRNLRGCYNQSEAGSHTLQS | | MYGCDVGPDGRLLRGHDQYA | |
| | MYGCDVGPDGRLLRGHDQYA | | YDGKDYIALNEDLRSWTAAD | |
| | YDGKDYIALNEDLRSWTAAD | | TAAQITQRKWEAAREAEQRR | |
| | TAAQITQRKWEAAREAEQRR | | AYLEGECVEWLRRYLENGKD | |
| | AYLEGECVEWLRRYLENGKD | | KLERADPPKTHVTHHPISDH | |
| | KLERADPPKTHVTHHPISDH | | EATLRCWALGFYPAEITLTW | |
| | EATLRCWALGFYPAEITLTW | | QRDGEDQTQDTELVETRPAG | |
| | QRDGEDQTQDTELVETRPAG | | DRTFQKWAAVVVPSGEEQRY | |
| | DRTFQKWAAVVVPSGEEQRY | | TCHVQHEGLPKPLTLRWEPS | |
| | TCHVQHEGLPKPLTLRWEPS | | SQST [symbol] PRGPTIKPCPPCKC | |
| | SQST [symbol] PRGPTIKPCPPCKC | | PAPNLLGGPSVFIFPPKIKD | |
| | PAPNLLGGPSVFIFPPKIKD | | VLMISLSPIVTCVVVDVSED | |
| | VLMISLSPIVTCVVVDVSED | | DPDVQISWFVNNVEVHTAQT | |
| | DPDVQISWFVNNVEVHTAQT | | QTHREDYNSTLRVVSALPIQ | |
| | QTHREDYNSTLRVVSALPIQ | | HQDWMSGKEFKCKVNNKDLP | |
| | HQDWMSGKEFKCKVNNKDLP | | APIERTISKPKGSVRAPQVY | |
| | APIERTISKPKGSVRAPQVY | | VLPPPEEEMTKKQVTLTCMV | |
| | VLPPPEEEMTKKQVTLTCMV | | TDFMPEDIYVEWTNNGKTEL | |
| | TDFMPEDIYVEWTNNGKTEL | | NYKNTEPVLDSDGSYFMYSK | |
| | NYKNTEPVLDSDGSYFMYSK | | LRVEKKNWVERNSYSCSVVH | |
| | LRVEKKNWVERNSYSCSVVH | | EGLHNHHTTKSFSRTPGK | |
| | EGLHNHHTTKSFSRTPGK | | | |
| #019 | MARSVTLVFLVLVSLTGLYA | 49 | GPMKLVMAFGCGASGGGGSG | 55 |
| (DVG9mer- | GPMKLVMAFGCGASGGGGSG | | GGGSIQRTPKIQVYSRHPAE | |
| B7-Fc) | GGGSIQRTPKIQVYSRHPAE | | NGKSNFLNCYVSGFHPSDIE | |
| | NGKSNFLNCYVSGFHPSDIE | | VDLLKNGERIEKVEHSDLSF | |
| | VDLLKNGERIEKVEHSDLSF | | SKDWSFYLLYYTEFTP1EKD | |
| | SKDWSFYLLYYTEFTP1EKD | | EYACRVNHVTLSQPKIVKWD | |
| | EYACRVNHVTLSQPKIVKWD | | RDMGGGGSGGGGSGGGGSGG | |
| | RDMGGGGSGGGGSGGGGSGG | | GGSGSHSMRYFYTSVSRPGR | |
| | GGSGSHSMRYFYTSVSRPGR | | GEPRFISVGYVDDTQFVRFD | |
| | GEPRFISVGYVDDTQFVRFD | | SDAASPREEPRAPWIEQEGP | |
| | SDAASPREEPRAPW1EQEGP | | EYWDRNTQIYKAQAQTDRES | |
| | EYWDRNTQIYKAQAQTDRES | | LRNLRGCYNQSEAGSHTLQS | |
| | LRNLRGCYNQSEAGSHTLQS | | MYGCDVGPDGRLLRGHDQYA | |
| | MYGCDVGPDGRLLRGHDQYA | | YDGKDYIALNEDLRSWTAAD | |
| | YDGKDYIALNEDLRSWTAAD | | TAAQITQRKWEAAREAEQRR | |
| | TAAQITQRKWEAAREAEQRR | | AYLEGECVEWLRRYLENGKD | |
| | AYLEGECVEWLRRYLENGKD | | KLERADPPKTHVTHHPISDH | |
| | KLERADPPKTHVTHHPISDH | | EATLRCWALGFYPAEITLTW | |
| | EATLRCWALGFYPAEITLTW | | QRDGEDQTQDTELVETRPAG | |
| | QRDGEDQTQDTELVETRPAG | | DRTFQKWAAVVVPSGEEQRY | |
| | DRTFQKWAAVVVPSGEEQRY | | TCHVQHEGLPKPLTLRWEPS | |
| | TCHVQHEGLPKPLTLRWEPS | | SQST [symbol] PRGPTIKPCPPCKC | |
| | SQST [symbol] PRGPTIKPCPPCKC | | PAPNLLGGPSVFIFPPKIKD | |
| | PAPNLLGGPSVFIFPPKIKD | | VLMISLSPIVTCVVVDVSED | |
| | VLMISLSPIVTCVVVDVSED | | DPDVQISWFVNNVEVHTAQT | |
| | DPDVQISWFVNNVEVHTAQT | | QTHREDYNSTLRVVSALPIQ | |
| | QTHREDYNSTLRVVSALPIQ | | HQDWMSGKEFKCKVNNKDLP | |
| | HQDWMSGKEFKCKVNNKDLP | | APIERTISKPKGSVRAPQVY | |
| | APIERTISKPKGSVRAPQVY | | VLPPPEEEMTKKQVTLTCMV | |
| | VLPPPEEEMTKKQVTLTCMV | | TDFMPEDIYVEWTNNGKTEL | |
| | TDFMPEDIYVEWTNNGKTEL | | NYKNTEPVLDSDGSYFMYSK | |
| | NYKNTEPVLDSDGSYFMYSK | | LRVEKKNWVERNSYSCSVVH | |
| | LRVEKKNWVERNSYSCSVVH | | EGLHNHHTTKSFSRTPGK | |
| | EGLHNHHTTKSFSRTPGK | | | |
| #020 | MARSVTLVFLVLVSLTGLYA | 50 | HPGFTILALGCGASGGGGSG | 56 |
| (DVH9mer- | HPGFTILALGCGASGGGGSG | | GGGSIQRTPKIQVYSRHPAE | |
| B7-Fc) | GGGSIQRTPKIQVYSRHPAE | | NGKSNFLNCYVSGFHPSDIE | |
| | NGKSNFLNCYVSGFHPSDIE | | VDLLKNGERIEKVEHSDLSF | |
| | VDLLKNGERIEKVEHSDLSF | | SKDWSFYLLYYTEFTPILKD | |
| | SKDWSFYLLYYTEFTPILKD | | EYACRVNHVTLSQPKIVKWD | |
| | EYACRVNHVTLSQPKIVKWD | | RDMGGGGSGGGGSGGGGSGG | |
| | RDMGGGGSGGGGSGGGGSGG | | GGSGSHSMRYFYTSVSRPGR | |
| | GGSGSHSMRYFYTSVSRPGR | | GEPRFISVGYVDDTQFVRFD | |
| | GEPRFISVGYVDDTQFVRFD | | SDAASPREEPRAPWIEQEGP | |
| | SDAASPREEPRAPW1EQEGP | | EYWDRNTQIYKAQAQTDRES | |
| | EYWDRNTQIYKAQAQTDRES | | LRNLRGCYNQSEAGSHTLQS | |
| | LRNLRGCYNQSEAGSHTLQS | | MYGCDVGPDGRLLRGHDQYA | |
| | MYGCDVGPDGRLLRGHDQYA | | YDGKDYIALNEDLRSWTAAD | |
| | YDGKDYIALNEDLRSWTAAD | | TAAQITQRKWEAAREAEQRR | |
| | TAAQITQRKWEAAREAEQRR | | AYLEGECVEWLRRYLENGKD | |
| | AYLEGECVEWLRRYLENGKD | | KLERADPPKTHVTHHPISDH | |
| | KLERADPPKTHVTHHPISDH | | EATLRCWALGFYPAEITLTW | |
| | EATLRCWALGFYPAEITLTW | | QRDGEDQTQDTELVETRPAG | |
| | QRDGEDQTQDTELVETRPAG | | DRTFQKWAAVVVPSGEEQRY | |
| | DRTFQKWAAVVVPSGEEQRY | | TCHVQHEGLPKPLTLRWEPS | |
| | TCHVQHEGLPKPLTLRWEPS | | SQST [symbol] PRGPTIKPCPPCKC | |
| | SQST [symbol] PRGPTIKPCPPCKC | | PAPNLLGGPSVFIFPPKIKD | |
| | PAPNLLGGPSVFIFPPKIKD | | VLMISLSPIVTCVVVDVSED | |
| | VLMISLSPIVTCVVVDVSED | | DPDVQISWFVNNVEVHTAQT | |
| | DPDVQISWFVNNVEVHTAQT | | QTHREDYNSTLRVVSALPIQ | |
| | QTHREDYNSTLRVVSALPIQ | | HQDWMSGKEFKCKVNNKDLP | |
| | HQDWMSGKEFKCKVNNKDLP | | APIERTISKPKGSVRAPQVY | |
| | APIERTISKPKGSVRAPQVY | | VLPPPEEEMTKKQVTLTCMV | |
| | VLPPPEEEMTKKQVTLTCMV | | TDFMPEDIYVEWTNNGKTEL | |
| | TDFMPEDIYVEWTNNGKTEL | | NYKNTEPVLDSDGSYFMYSK | |
| | NYKNTEPVLDSDGSYFMYSK | | LRVEKKNWVERNSYSCSVVH | |
| | LRVEKKNWVERNSYSCSVVH | | EGLHNHHTTKSFSRTPGK | |
| | EGLHNHHTTKSFSRTPGK | | | |

Accordingly, in various embodiments, the fusion protein can comprise an amino acid sequence having at least 85%, at least 90%, at least 95%, or at least 99% sequence identity to any one of SEQ ID NOs: 45-56 (Table 7). For example, the fusion protein can comprise an amino acid sequence comprising any one of SEQ ID NOs: 45-56.

Further, the fusion protein can comprise an amino acid sequence consisting or consisting essentially of any one of SEQ ID NOs: 45-56. Preferably, the fusion protein comprises any one of SEQ ID NOs: 45,48-51, and 54-56. For example, the fusion protein can comprise an amino acid sequence consisting or consisting essentially of any one of SEQ ID NOs: 45,48-51, and 54-56.

### Fusion Proteins Lacking the Antigen Peptide

Also provided are fusion proteins as described above but lacking the antigen peptide and the first flexible linker. Accordingly, a second fusion protein is provided comprising a β2-microglobulin, a flexible linker, a MHC heavy chain and a fragment crystallizable (Fc) portion of an antibody. In various embodiments, the β2-microglobulin may comprise a β2 microglobulin as described above (such as a human or murine β2 microglobulin). In various embodiments, the flexible linker may be the second flexible linker described above (e.g., may comprise SEQ ID NO: 35 or 36). In further embodiments, the MHC heavy chain may comprise the MHC heavy chains described above. The Fc portion may also comprise the Fc portion described above.

Also provided are fusion protein complexes comprising the second fusion protein described herein and an antigen peptide. The antigen peptide can be any antigen peptide described above. Preferably, the antigen peptide complexes with the second fusion protein through (e.g., affinity bonding or a non-covalent linkage). In various embodiments, the antigen peptide stabilizes the fusion protein complex.

### Function and Binding Affinity of the Fusion Proteins

In various embodiments, the fusion protein or fusion protein complex described herein can bind to a cell surface receptor having an affinity for a MHC-peptide complex. Resting B-cells that will eventually express an antibody having a particular antigen binding site also express a cell-surface antibody/immunoglobulin that contains the same antigen binding site. These are called “B-cell receptors”. Consequently, the cell surface receptor having an affinity for a MI-IC-peptide complex can be a B-cell receptor expressed on the surface of a hybridoma or B-cell. Likewise, the fusion protein or fusion protein complex may bind specifically to anti-MHC antibodies (e.g., anti-HLA antibodies) secreted by those hybridomas or B-cells. Further, resting T-cells also express T-cell receptors having an affinity for a MHC-peptide complex and in the context of tissue grafting, this receptor may be specific for a foreign MHC-peptide complex (e.g., see Amir et al., Blood 2011; 118:6733-42, incorporated herein by reference in its entirety). Consequently, the cell surface receptor having an affinity for a MHC-peptide complex can be a T-cell receptor.

In certain embodiments, the fusion proteins are provided as dimers comprising two fusion proteins wherein the Fc portions of the constructs are covalently linked (for example, by one or more disulfide bonds). In this way, an antibody-like structure is generated where the antibody variable region (e.g., an “Fab” region) is replaced with the SCT constructs described herein. See, e.g., FIG. 1 for an illustration of a dimer formed by the linkage of two fusion proteins described herein as compared to a traditional antibody-like structure.

### Nucleic Acids

A nucleic acid is provided, the nucleic acid comprising a nucleotide sequence encoding the fusion protein described herein. The skilled artisan will appreciate that functional variants of these nucleic acid molecules are also intended to be a part of the present invention. Functional variants are nucleic acid sequences that can be directly translated, using the standard genetic code, to provide an amino acid sequence identical to that translated from the parental nucleic acid molecules.

For ease of reference, Table 8 provides illustrative nucleic acids for encoding some of the components of the fusion protein as described above. Each of the nucleotide sequences provided in Table 8 encodes a peptide or protein having an amino acid sequence described above. The SEQ ID NOs of the amino acid sequences encoded by each of the nucleic acid sequences provided in Table 8 are also indicated.

Further, in Table 8, the nucleotide sequence encoding the murine IgG2-Fc domain (SEQ ID NO: 68) is provided without a nucleotide sequence (AGATCT) at its amino terminus that is included in the nucleic acid transcripts for the full-length protein (Table 9 below). This nucleotide sequence corresponds to a BgIII restriction site and encodes two residues (D-S) in the complete fusion protein as annotated in Table 7 (SEQ ID NOs: 45-56). These residues are not considered part of the Fc domain so the nucleotide sequence AGATCT is omitted from SEQ ID NO: 68.

TABLE 8

| | | Amino | | Nucleic |
|---|---|---|---|---|
| Fusion | | Acid | | Acid |
| Protein | | SEQ ID | | SEQ ID |
| Part | Name | NO | Nucleic Acid Sequence | NO: |
| Leader | N/A | 44 | ATGGCTCGCTCGGTGACCCTGGTCTTT | 57 |
| | | | CTGGTGCTTGTCTCACTGACCGGTTTG | |
| | | | TATGCT | |
| Antigen | CMVpp65 | 1 | AACCTGGTGCCCATGGTGGCCACCGTG | 58 |
| Peptide | | | | |
| | Vaccinia virus | 2 | CTGCCCTGCCAGCTGATGTACGCCCTG | 59 |
| | (L-9mer) | | | |
| | Dengue virus | 3 | GGCCCCATGAAGCTGGTGATGGCCTTC | 60 |
| | (G-9mer) | | | |
| | Dengue virus | 4 | CACCCCGGCTTCACCATCCTGGCCCTG | 61 |
| | (H-9mer) | | | |
| Linkers | Linkerl | 38 | GGATGCGGTGCTAGCGGTGGTGGAGGT | 62 |
| | | | AGCGGAGGTGGAGGAAGC | |
| | Linker2 | 35 | GGCGGTGGTGGTTCCGGTGGAGGCGGT | 63 |
| | | | TCCGGAGGTGGTGGATCCGGTGGTGGA | |
| | | | GGTAGT | |
| Human β2- | hb2m | 24 | ATCCAGCGTACTCCAAAGATTCAGGTT | 64 |
| micro- | | | TACTCACGTCATCCAGCAGAGAATGGA | |
| globulin | | | AAGTCAAATTTCCTGAATTGCTATGTG | |
| | | | TCTGGGTTTCATCCATCCGACATTGAA | |
| | | | GTTGACTTACTGAAGAATGGAGAGAGA | |
| | | | ATTGAAAAAGTGGAGCATTCAGACTTG | |
| | | | TCTTTCAGCAAGGACTGGTCTTTCTAT | |
| | | | CTCTTGTACTACACTGAATTCACCCCC | |
| | | | ACTGAAAAAGATGAGTATGCCTGCCGT | |
| | | | GTGAACCATGTGACTTTGTCACAGCCC | |
| | | | AAGATAGTTAAGTGGGATCGAGACATG | |
| HLA | HLA-A*02:01 | 25 | GGCTCTCACTCCATGAGGTATTTCTTC | 65 |
| | mutated_Y84C | | ACATCCGTGTCCCGGCCCGGCCGCGGG | |
| | | | GAGCCCCGCTTCATCGCAGTGGGCTAC | |
| | | | GTGGACGACACGCAGTTCGTGCGGTTC | |
| | | | GACAGCGACGCCGCGAGCCAGAGGATG | |
| | | | GAGCCGCGGGCGCCGTGGATAGAGCAG | |
| | | | GAGGGTCCGGAGTATTGGGACGGGGAG | |
| | | | ACACGGAAAGTGAAGGCCCACTCACAG | |
| | | | ACTCACCGAGTGGACCTGGGGACCCTG | |
| | | | CGCGGCTGCTACAACCAGAGCGAGGCC | |
| | | | GGTTCTCACACCGTCCAGAGGATGTAT | |
| | | | GGCTGCGACGTGGGGTCGGACTGGCGC | |
| | | | TTCCTCCGCGGGTACCACCAGTACGCC | |
| | | | TACGACGGCAAGGATTACATCGCCCTG | |
| | | | AAAGAGGACCTGCGCTCTTGGACCGCG | |
| | | | GCGGACATGGCAGCTCAGACCACCAAG | |
| | | | CACAAGTGGGAGGCGGCCCATGTGGCG | |
| | | | GAGCAGTTGAGAGCCTACCTGGAGGGC | |
| | | | ACGTGCGTGGAGTGGCTCCGCAGATAC | |
| | | | CTGGAGAACGGGAAGGAGACGCTGCAG | |
| | | | CGCACGGACGCCCCCAAAACGCATATG | |
| | | | ACTCACCACGCTGTCTCTGACCATGAA | |
| | | | GCCACCCTGAGGTGCTGGGCCCTGAGC | |
| | | | TTCTACCCTGCGGAGATCACACTGACC | |
| | | | TGGCAGCGGGATGGGGAGGACCAGACC | |
| | | | CAGGACACGGAGCTCGTGGAGACCAGG | |
| | | | CCTGCAGGGGATGGAACCTTCCAGAAG | |
| | | | TGGGCGGCTGTGGTGGTGCCTTCTGGA | |
| | | | CAGGAGCAGAGATACACCTGCCATGTG | |
| | | | CAGCATGAGGGTTTGCCCAAGCCCCTC | |
| | | | ACCCTGAGATGGGAGCCGTCTTCCCAG | |
| | | | CCCACC | |
| | HLA-A*11:01 | 27 | GGCTCCCACTCCATGAGGTATTTCTAC | 66 |
| | mutated_Y84C | | ACCTCCGTGTCCCGGCCCGGCCGCGGG | |
| | | | GAGCCCCGCTTCATCGCCGTGGGCTAC | |
| | | | GTGGACGACACGCAGTTCGTGCGGTTC | |
| | | | GACAGCGACGCCGCGAGCCAGAGGATG | |
| | | | GAGCCGCGGGCGCCGTGGATAGAGCAG | |
| | | | GAGGGGCCGGAGTATTGGGACCAGGAG | |
| | | | ACACGGAATGTGAAGGCCCAGTCACAG | |
| | | | ACTGACCGAGTGGACCTGGGGACCCTG | |
| | | | CGCGGCTGCTACAACCAGAGCGAGGAC | |
| | | | GGTTCTCACACCATCCAGATAATGTAT | |
| | | | GGCTGCGACGTGGGGCCGGACGGGCGC | |
| | | | TTCCTCCGCGGGTATCGGCAGGACGCC | |
| | | | TACGACGGCAAGGATTACATCGCCCTG | |
| | | | AACGAGGACCTGCGCTCTTGGACCGCG | |
| | | | GCGGACATGGCAGCTCAGATCACCAAG | |
| | | | CGCAAGTGGGAGGCGGCCCATGCGGCG | |
| | | | GAGCAGCAGAGAGCCTACCTGGAGGGC | |
| | | | CGGTGCGTGGAGTGGCTCCGCAGATAC | |
| | | | CTGGAGAACGGGAAGGAGACGCTGCAG | |
| | | | CGCACGGACCCCCCCAAGACACATATG | |
| | | | ACCCACCACCCCATCTCTGACCATGAG | |
| | | | GCCACCCTGAGGTGCTGGGCCCTGGGC | |
| | | | TTCTACCCTGCGGAGATCACACTGACC | |
| | | | TGGCAGCGGGATGGGGAGGACCAGACC | |
| | | | CAGGACACGGAGCTCGTGGAGACCAGG | |
| | | | CCTGCAGGGGATGGAACCTTCCAGAAG | |
| | | | TGGGCGGCTGTGGTGGTGCCTTCTGGA | |
| | | | GAGGAGCAGAGATACACCTGCCATGTG | |
| | | | CAGCATGAGGGTCTGCCCAAGCCCCTC | |
| | | | ACCCTGAGATGGGAGCTGTCTTCCCAG | |
| | | | CCCACC | |
| | HLA-B*07:02 | 29 | GGCTCCCACTCCATGAGGTATTTCTAC | 67 |
| | mutated_Y84C | | ACCTCCGTGTCCCGGCCCGGCCGCGGG | |
| | | | GAGCCCCGCTTCATCTCAGTGGGCTAC | |
| | | | GTGGACGACACCCAGTTCGTGAGGTTC | |
| | | | GACAGCGACGCCGCGAGTCCGAGAGAG | |
| | | | GAGCCGCGGGCGCCGTGGATAGAGCAG | |
| | | | GAGGGGCCGGAGTATTGGGACCGGAAC | |
| | | | ACACAGATATACAAGGCCCAGGCACAG | |
| | | | ACTGACCGAGAGAGCCTGCGGAACCTG | |
| | | | CGCGGCTGCTACAACCAGAGCGAGGCC | |
| | | | GGGTCTCACACCCTCCAGAGCATGTAC | |
| | | | GGCTGCGACGTGGGGCCGGACGGGCGC | |
| | | | CTCCTCCGCGGGCATGACCAGTACGCC | |
| | | | TACGACGGCAAGGATTACATCGCCCTG | |
| | | | AACGAGGACCTGCGCTCCTGGACCGCC | |
| | | | GCGGACACGGCGGCTCAGATCACCCAG | |
| | | | CGCAAGTGGGAGGCGGCCCGTGAGGCG | |
| | | | GAGCAGCGGAGAGCCTACCTGGAGGGC | |
| | | | GAGTGCGTGGAGTGGCTCCGCAGATAC | |
| | | | CTGGAGAACGGGAAGGACAAGCTGGAG | |
| | | | CGCGCTGACCCCCCAAAGACACACGTG | |
| | | | ACCCACCACCCCATCTCTGACCATGAG | |
| | | | GCCACCCTGAGGTGCTGGGCCCTGGGT | |
| | | | TTCTACCCTGCGGAGATCACACTGACC | |
| | | | TGGCAGCGGGATGGCGAGGACCAAACT | |
| | | | CAGGACACTGAGCTTGTGGAGACCAGA | |
| | | | CCAGCAGGAGATAGAACCTTCCAGAAG | |
| | | | TGGGCAGCTGTGGTGGTGCCTTCTGGA | |
| | | | GAAGAGCAGAGATACACATGCCATGTA | |
| | | | CAGCATGAGGGGCTGCCGAAGCCCCTC | |
| | | | ACCCTGAGATGGGAGCCGTCTTCCCAG | |
| | | | TCCACC | |
| Fc | mIgG2a-Fc | 42 | CCCAGAGGGCCCACAATCAAGCCCTGT | 68 |
| | | | CCTCCATGCAAATGCCCAGCACCTAAC | |
| | | | CTCTTGGGTGGACCATCCGTCTTCATC | |
| | | | TTCCCTCCAAAGATCAAGGATGTACTC | |
| | | | ATGATCTCCCTGAGCCCCATAGTCACA | |
| | | | TGTGTGGTGGTGGATGTGAGCGAGGAT | |
| | | | GACCCAGATGTCCAGATCAGCTGGTTT | |
| | | | GTGAACAACGTGGAAGTACACACAGCT | |
| | | | CAGACACAAACCCATAGAGAGGATTAC | |
| | | | AACAGTACTCTCCGGGTGGTCAGTGCC | |
| | | | CTCCCCATCCAGCACCAGGACTGGATG | |
| | | | AGTGGCAAGGAGTTCAAATGCAAGGTC | |
| | | | AACAACAAAGACCTCCCAGCGCCCATC | |
| | | | GAGAGAACCATCTCAAAACCCAAAGGG | |
| | | | TCAGTAAGAGCTCCACAGGTATATGTC | |
| | | | TTGCCTCCACCAGAAGAAGAGATGACT | |
| | | | AAGAAACAGGTCACTCTGACCTGCATG | |
| | | | GTCACAGACTTCATGCCTGAAGACATT | |
| | | | TACGTGGAGTGGACCAACAACGGGAAA | |
| | | | ACAGAGCTAAACTACAAGAACACTGAA | |
| | | | CCAGTCCTGGACTCTGATGGTTCTTAC | |
| | | | TTCATGTACAGCAAGCTGAGAGTGGAA | |
| | | | AAGAAGAACTGGGTGGAAAGAAATAGC | |
| | | | TACTCCTGTTCAGTGGTCCACGAGGGT | |
| | | | CTGCACAATCACCACACGACTAAGAGC | |
| | | | TTCTCCCGGACTCCGGGTAAATGA | |

Illustrative nucleic acid sequences that can be used to encode the fusion proteins described herein are identified by their SEQ ID NO in Table 9, below. Note that each sequence encodes an amino acid chain identified above in Table 6. For ease of reference, the SEQ ID NO that corresponds to the encoded amino acid for each nucleic acid sequence is also provided.

TABLE 9

| Fusion Protein Name | Nucleic Acid | Amino Acid |
|---|---|---|
| #006 (CMVpp65-A2-Fc) | SEQ ID NO: 69 | SEQ ID NO: 45 |
| #007 (CMVpp65-A2-Fc_{LALAPG}) | SEQ ID NO: 70 | SEQ ID NO: 46 |
| #010 (CMVpp65-B7-Fc) | SEQ ID NO: 71 | SEQ ID NO: 47 |
| #018 (VVL9mer-B7-Fc) | SEQ ID NO: 72 | SEQ ID NO: 48 |
| #019 (DVG9mer-B7-Fc) | SEQ ID NO: 73 | SEQ ID NO: 49 |
| #020 (DVH9mer-B7-Fc) | SEQ ID NO: 74 | SEQ ID NO: 50 |

Accordingly, the nucleic acid provided herein may comprise a nucleotide sequence comprising one or more of SEQ ID NOs: 57-74. For example, the nucleic acid may comprise a nucleotide sequence comprising any one of SEQ ID NOs: 69-74. In various embodiments, the nucleic acid consists or consists essentially of any one of SEQ ID NOs: 69-74.

By “encoding” or “encoded”, with respect to a specified nucleic acid, is meant comprising the information for translation into the specified protein. A nucleic acid encoding a protein may comprise intervening sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. When the nucleic acid is prepared or altered synthetically, advantage can be taken of known codon preferences of the intended host where the nucleic acid is to be expressed.

The nucleic acid may comprise cDNA.

In various embodiments, a nucleic acid encoding the fusion protein can be prepared, in a first step, by ligation of sequences encoding the MHC heavy chain and the β2-microglobulin to a sequence encoding an antigen peptide to form a nucleic acid encoding the SCT portion of the fusion protein. This resulting SCT encoding nucleic acid can then be ligated to a nucleotide sequence encoding an Fc portion of an antibody to generate a vector encoding the complete fusion protein described herein. Such vectors may comprise, for example, the nucleic acids described in Table 8 and 9 above.

DNA encoding the antigen peptide can be obtained by isolating DNA from natural sources or by known synthetic methods, e.g., the phosphate tri-ester method (see e.g., Oligonucleotide Synthesis, IRL Press, M. Gait, ed., 1984). In some aspects, DNA encoding a class I heavy chain can be obtained from a suitable cell line such as, for example, human lymphoblastoid cells. In various configurations, a gene or cDNA encoding a class I heavy chain can be amplified by the polymerase chain reaction (PCR) or other means known in the art. In some aspects, a PCR product can also include sequences encoding linkers, and/or one or more restriction enzyme sites for ligation of such sequences. Synthetic oligonucleotides can also be prepared using commercially available automated oligonucleotide synthesizers. DNA sequences encoding flexible linkers (e.g., the first and second flexible linkers described above) can be interposed between a β2-microglobulin segment and a sequence encoding an antigen peptide segment, and interposed between a β2-microglobulin segment and the heavy chain segment. In some embodiments, the segments can be joined using a ligase.

The nucleic acids provided herein may further comprise additional nucleotide sequences. For example, a promoter sequence, which controls expression of the sequence coding for the β2-microglobulin segment covalently bound to the peptide ligand segment, and a sequence encoding a leader peptide (which can direct the fusion protein to the cell surface or the culture medium) can also be included in the nucleic acid or be present in the expression vector into which the nucleic acid is inserted. In a non-limiting example, an immunoglobulin or CMV promoter can be used for expression of the fusion protein described herein. A strong translation initiation sequence can also be included in the construct to enhance efficiency of translational initiation, such as, for example, the Kozak consensus sequence (CCACCATG) or an internal ribosome entry site (IRES). In some configurations, the nucleic acid provided herein encoding for the fusion protein can further encode an amino terminal leader peptide. When expressed in a host cell, the primary translation product of such a nucleic acid can comprise a leader peptide which can be removed by the host cell posttranslationally.

In some configurations, a leader sequence encoding the leader peptide and contained in the nucleic acid herein can further comprise one or more restriction sites so that an oligonucleotide encoding an antigen peptide segment of interest can be attached to the first linker. In some aspects, a restriction site can be incorporated into the 3′ end of the DNA sequence encoding a leader peptide sequence and can be, for example, 2 to 10 codons in length, and can be positioned before the coding region for the peptide ligand. A non-limiting example of a restriction site is the AfIII site, although other cleavage sites also can be incorporated before the peptide ligand coding region. As discussed herein, use of such a restriction site in combination with a second restriction site, typically positioned at the beginning of the sequence coding for the linker can allow rapid and straightforward insertion of sequences coding for a wide variety of peptide ligands into a DNA construct encoding the fusion protein.

Accordingly, an expression vector is also provided. The expression vector comprises one or more of the nucleic acids described herein. A vector is capable of transporting a nucleic acid molecule to which it has been linked. Cloning as well as expression vectors are contemplated by the term “vector”, as used herein. Vectors include, but are not limited to, plasmids, cosmids, bacterial artificial chromosomes (BAC) and yeast artificial chromosomes (YAC) and vectors derived from bacteriophages or plant or animal (including human) viruses. Vectors comprise an origin of replication recognized by the proposed host and in case of expression vectors, promoter and other regulatory regions recognized by the host. A vector containing a second nucleic acid molecule can be introduced into a cell by transformation, transfection, or by making use of viral entry mechanisms. Certain vectors are capable of autonomous replication in a host into which they are introduced (e.g., vectors having a bacterial origin of replication can replicate in bacteria). Other vectors can be integrated into the genome of a host upon introduction into the host, and thereby are replicated along with the host genome.

Vectors can be derived from plasmids such as: F, F1, RP1, Col, pBR322, TOL, Ti, etc; cosmids; phages such as lambda, lambdoid, M13, Mu, P1, P22, Qβ, T-even, T-odd, T2, T4, T7, etc.; or plant viruses. Vectors can be used for cloning and/or expression of the antibodies or antigen-binding fragments of the invention and might even be used for gene therapy purposes. Vectors comprising one or more nucleic acid molecules according to the invention operably linked to one or more expression-regulating nucleic acid molecules are also covered by the present invention. The choice of the vector is dependent on the recombinant procedures followed and the host used. Introduction of vectors in host cells can be effected by inter alia calcium phosphate transfection, virus infection, DEAE-dextran mediated transfection, lipofectamine transfection or electroporation. Vectors may be autonomously replicating or may replicate together with the chromosome into which they have been integrated. Preferably, the vectors contain one or more selection markers. The choice of the markers may depend on the host cells of choice. They include, but are not limited to, kanamycin, neomycin, puromycin, hygromycin, zeocin, the thymidine kinase gene from Herpes simplex virus (HSV-TK), and the dihydrofolate reductase gene from mouse (dhfr). Vectors comprising one or more nucleic acid molecules encoding the heavy and light variable chains as described above operably linked to one or more nucleic acid molecules encoding proteins or peptides that can be used to isolate the human binding molecules are also covered by the invention. These proteins or peptides include, but are not limited to, glutathione-S-transferase, maltose binding protein, polyhistidine, green fluorescent protein, luciferase and beta-galactosidase.

The term “operably linked” refers to two or more nucleic acid sequence elements that are usually physically linked and are in a functional relationship with each other. For instance, a promoter is operably linked to a coding sequence, if the promoter is able to initiate or regulate the transcription or expression of a coding sequence, in which case the coding sequence should be understood as being “under the control of” the promoter.

The expression vector may be transfected into a host cell to induce the translation and expression of the nucleic acid into the heavy chain variable region and/or the light chain variable region. Therefore, a host cell is provided comprising any expression vector described herein. Host cells include, but are not limited to, cells of mammalian, plant, insect, fungal or bacterial origin. Bacterial cells include, but are not limited to, cells from Gram-positive bacteria or Gram-negative bacteria such as several species of the genera Escherichia, such as E. coli, and Pseudomonas. In the group of fungal cells preferably yeast cells are used. Expression in yeast can be achieved by using yeast strains such as inter alia Pichia pastoris, Saccharomyces cerevisiae and Hansenula polymorpha. Furthermore, insect cells such as cells from Drosophila and Sf9 can be used as host cells. Besides that, the host cells can be plant cells such as, inter alia, cells from crop plants such as forestry plants, or cells from plants providing food and raw materials such as cereal plants, or medicinal plants, or cells from ornamentals, or cells from flower bulb crops. Transformed (transgenic) plants or plant cells are produced by known methods, for example, Agrobacterium-mediated gene transfer, transformation of leaf discs, protoplast transformation by polyethylene glycol-induced DNA transfer, electroporation, sonication, microinjection or biolistic gene transfer. Additionally, a suitable expression system can be a baculovirus system.

Expression systems using mammalian cells, such as Chinese Hamster Ovary (CHO) cells, COS cells, J558 cells, SP2-O cells BHK cells, NSO cells or Bowes melanoma cells are preferred in the present invention. Mammalian cells provide expressed proteins with posttranslational modifications that are most similar to natural molecules of mammalian origin. Since the present invention deals with molecules that may have to be administered to humans, a completely human expression system would be particularly preferred. Therefore, even more preferably, the host cells are human cells. Examples of suitable human cells are inter alia HeLa, 911, AT1080, A549, HEK293, and HEK293T.

In various configurations, cells expressing the fusion protein described herein can be identified using known methods. For example, expression of the fusion protein can be determined by an ELISA or Western blot using an antibody probe against the MHC heavy chain or the Fc portion of the fusion protein.

An expressed fusion protein can be isolated and purified by known methods. For example, affinity purification using Sepharose columns can be used according to general procedures known in the art (e.g., see Harlow E. et al., Antibodies, A Laboratory Manual (1988). Further, the fusion protein can also contain a sequence to aid in purification such as a 6×His tag. Additional details on molecular engineering tools and techniques that may be used to generate the disclosed fusion proteins are described below.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “fusion protein” as used herein refers to a protein having a polypeptide sequence that comprises sequences derived from two or more separate proteins. A fusion protein can be generated by joining together a nucleic acid molecule that encodes all or part of a first polypeptide with a nucleic acid molecule that encodes all or part of a second polypeptide to create a nucleic acid sequence which, when expressed, yields a single polypeptide having functional properties derived from each of the original proteins.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

Constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y*100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tm=81.5° C.+16.6(log 10[Na+])+0.41(fraction G/C content)−0.63(% formamide)−(600/1). Furthermore, the Tm of a DNA:DNA hybrid is decreased by 1-1.5° C. for every 1% decrease in nucleotide identity (see e.g., Sambrook and Russel, 2006).

Host cells can be transformed using a variety of standard techniques (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain | |
|---|---|
| Characteristic | Amino Acid |
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged | D E |
| (Acidic): | |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Pharmaceutical Compositions

Also provided are pharmaceutical compositions. The pharmaceutical compositions comprise at least one fusion protein described herein and a pharmaceutically acceptable carrier. In various embodiments the pharmaceutical compositions comprise two or more fusion proteins or dimers thereof.

The compositions provided herein can comprise a pharmaceutically acceptable carrier. As used herein, a pharmaceutically acceptable carrier is inclusive of any pharmaceutically acceptable excipients. The “pharmaceutically acceptable excipient” is an excipient that is non-toxic to recipients at the used dosages and concentrations, and is compatible with other ingredients of the formulation comprising the drug, agent or binding molecule. The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Pharmaceutical compositions provided herein can comprise a therapeutically effective amount of the fusion protein described herein, which can be in purified form, together with a suitable amount of carrier or excipient so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, subcutaneous, intranasal, epidural, ophthalmic, transdermal, buccal, and rectal. In various embodiments, the agents of use may be formulated for administration by injection or infusion. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic or Research Methods

Provided herein are methods of depleting a population of antigen specific cells expressing a surface receptor having an affinity for a MHC-peptide complex, the method comprising contacting the cells with an effective amount of the fusion protein or fusion protein complex described herein.

In various embodiments, the antigen specific cells may be in vitro (that is, cultured in a dish or artificial environment). In certain embodiments, the method can further comprise contacting the cells with complement (e.g., a C1 complex or components thereof). This can initiate the complement cascade and trigger the degradation of the targeted cells (that is, those expressing the surface receptor having an affinity for the MHC-peptide complex).

Alternatively, the antigen specific cells may be located in vivo (e.g., in a subject in need of depleting a population of antigen specific cells). In this case, the method comprises administering a pharmaceutical acceptable amount of the fusion protein or fusion protein complex. In some embodiments, for example, the fusion protein may be administered as a dimer as described herein. In some embodiments, the fusion protein or fusion protein complex may be administered in a suitable pharmaceutical composition or formulation as described herein.

In various embodiments, the MHC type I heavy chain of the fusion protein or fusion protein complex comprises the heavy chain of the MHC-peptide complex having an affinity for the surface receptor of the antigen-specific cells. In certain embodiments, the antigen peptide of the fusion protein or fusion protein complex comprises the peptide of the MHC-peptide complex having an affinity for the surface receptor of the antigen-specific cells. In various embodiments the MHC type I heavy chain of the fusion protein or fusion protein complex comprises the heavy chain of the MHC-peptide complex and the peptide of the fusion protein or fusion protein complex comprises the peptide of the MHC-peptide complex having an affinity for the surface receptor of the antigen-specific cells.

The administration or delivery of the fusion proteins or fusion protein complexes described herein can advantageously target any cell that expresses a surface receptor having a specific affinity for the MHC-peptide complex embodied by the fusion protein or complex. The two major cell populations in the adaptive immune system (T-cells and B-cells) both express similar antigen recognizing receptors called T-cell receptors and B cell receptors, respectively. Therefore, suitable cell populations to target using the methods described herein include T-cells and B-cells. For example, when the cells are antigen-specific B cells the cell surface receptor can comprise a B-cell receptor. When the cells are antigen-specific T cells, the cell surface receptor can comprise a T-cell receptor.

In various embodiments, the MHC-peptide complex targeted by the cell surface receptors of the cell population is a HLA-peptide complex. In certain embodiments, the HLA-peptide complex can comprise a foreign and/or allotypic HLA-peptide complex (that is, a complex comprising a non-native HLA component to the subject). As used herein, the terms “allotype” or “allotypic” or “foreign” each refers to a different version of a MHC complex that is expressed in a separate member belonging to the same species as the subject.

T-cells are largely responsible for eliciting the immune response underlying graft versus host disease and have been considered to be sensitive to allotypic MHC proteins (e.g., allo-HLA) expressed on host tissue. Recently, it has been found that they also show high specificity for specific allo-HLA-peptide complexes (see Amir et al., Blood 2011; 118:6733-42; incorporated herein by reference in its entirety). This makes them a promising target for the fusion proteins or fusion protein complexes described herein. Accordingly, a method is provided for depleting a population of T cells in a subject in need thereof (e.g., a subject suffering from graft-versus-host-disease), the method comprising administering a therapeutic amount of the fusion protein or fusion protein complex described herein to the subject.

B-cells are responsible for synthesizing and secreting antibodies to foreign antibodies, but each also expresses a cell surface receptor (B-cell receptor) that contains an immunoglobulin domain that mirrors the antibody it produces. Therefore, a B-cell population specific for given MHC-peptide complex can be depleted (along with its corresponding antibodies) by targeting its B-cell receptors using the fusion proteins or fusion protein complex described herein. As explained below, this may be particularly useful in treating diseases and conditions such as antibody-mediated transplant rejection, organ transplant rejection, blood transfusion refractoriness, or antibody-mediated hemolysis.

Accordingly, a method is provided for treating antibody-mediated transplant rejection in a subject in need thereof, wherein the antibody-mediated rejection is caused by antibodies having an affinity for a foreign HLA-peptide complex, the method comprising depleting a population of B cells that express a surface receptor having an affinity for the foreign HLA-peptide complex in the subject according to the methods described herein above.

For example, a method for treating organ transplant rejection, antibody mediated rejection, graft-versus host disease, and/or blood transfusion refractoriness in a subject in need thereof is provided, the method comprising administering the fusion protein or fusion protein complex described herein to the subject. In various embodiments, the subject in need thereof produces antibodies having an affinity for a foreign HLA-peptide complex and wherein administering the fusion protein depletes a population of B cells in the subject that express the antibodies. In still further embodiments, administering the fusion protein or fusion protein complex depletes a population of T cells in the subject expressing a T-cell receptor (TCR) having an affinity for the foreign HLA-peptide complex.

Advantageously, the methods described herein allow for the depletion of a specific immune cell population (e.g., a T-cell population and/or a B cell population) without impairing global humoral immunity.

Also provided is a method of treating antibody mediated hemolysis in a subject in need thereof, the method comprising administering the fusion protein or the fusion protein complex to the subject.

Methods described herein are generally performed on a subject in need thereof. A subject in need of the therapeutic methods described herein can be a subject having, diagnosed with, suspected of having, or at risk for developing a B-cell or T-cell mediated disease, disorder, or condition. Specifically, the subject will preferably be diagnosed with, suspected of having, or at risk of developing an immune response against a foreign MHC complex (e.g., an allotypic HLA) such as, for example, graft, transplant or transfusion candidates. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

In various embodiments, the fusion protein may be administered as a dimer comprising two fusion proteins linked by one or more disulfide bonds. Further, the fusion protein or dimer thereof or fusion protein complex may be administered as part of a pharmaceutically acceptable composition as described herein above.

Generally, a safe and effective amount of the fusion protein is, for example, that amount that would cause the desired therapeutic effect in a subject while minimizing undesired side effects. In various embodiments, an effective amount of the fusion protein described herein can substantially deplete B-cells with antigenic specificity and without inhibiting global humoral immunity.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of the fusion protein can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to deplete B-cells with antigenic specificity and without inhibiting global humoral immunity.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a fusion protein can occur as a single event or over a time course of treatment. For example, a fusion protein can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a B-cell mediated disease, disorder, or condition.

A fusion protein can be administered simultaneously or sequentially with another agent, such as an antibiotic, an anti-inflammatory, or another agent. For example, a fusion protein can be administered simultaneously with another agent, such as an antibiotic or an anti-inflammatory. Simultaneous administration can occur through administration of separate compositions, each containing one or more of a fusion protein, an antibiotic, an anti-inflammatory, or another agent.

### Administration

Agents and compositions described herein can be administered according to methods described herein in a variety of means. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8A to about 15A.

### Imaging Agents and Uses Thereof

Also provided are imaging agents and methods of use thereof. The imaging agents provided herein can comprise a fusion protein conjugated to a signaling moiety. The signaling moiety can be any signaling generating moiety known in the art (e.g., a fluorophore, a fluorochrome, a radioisotope, a positron emitting isotope, or any combination thereof).

Accordingly, a method is also provided for staining an antigen specific cell population, the method comprising contacting the antigen specific cells with an imaging agent described herein. Preferably, the antigen specific cell population are cells expressing a surface receptor that has an affinity for a particular MHC-peptide complex (e.g., a HLA-peptide complex). In various embodiments, the imaging agent comprises a fusion protein that contains a MHC-peptide complex targeted by the surface receptor.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Sequences Used in the Examples

For ease of reference, the DNA and amino acid sequences used to synthesize the fusion proteins in the following examples are described in Table 10 below, along with their SEQ ID NOs. Sequences corresponding to the complete fusion protein transcripts are listed in Table 11. Other sequences not included in these tables are described as applicable in the following examples.

TABLE 10
Fusion Protein Components

| Fusion Protein or | | Nucleic Acid | Amino Acid |
|---|---|---|---|
| Component | Name | SEQ ID NO: | SEQ ID NO: |
| Leader | N/A | 57 | 44 |
| Antigen Peptide | CMVpp65 | 58 | 1 |
| | Vaccinia virus | 59 | 2 |
| | (L-9mer) | | |
| | Dengue virus | 60 | 3 |
| | (G-9mer) | | |
| | Dengue virus | 61 | 4 |
| | (H-9mer) | | |
| Linkers | Linker1 | 62 | 38 |
| | Linker2 | 63 | 35 |
| Human β2-microglob-ulin | hb2m | 64 | 24 |
| HLA | HLA-A*02:01 | 65 | 25 |
| | HLA-A*11:01 | 66 | 27 |
| | HLA-B*07:02 | 67 | 29 |
| Fc | mIgG2-Fc | 68 | 42 |

TABLE 11
Fusion Protein Constructs

| | Nucleic Acid | Amino Acid |
|---|---|---|
| | SEQ ID NO: | SEQ ID NO: |
| #006 (CMVpp65-A2-Fc) | 69 | 45 |
| #007 (CMVpp65-A2-Fc_{LALAPG}) | 70 | 46 |
| #010 (CMVpp65-B7-Fc) | 71 | 47 |
| #018 (VVL9mer-B7-Fc) | 72 | 48 |
| #019 (DVG9mer-B7-Fc) | 73 | 49 |
| #020 (DVH9mer-B7-Fc) | 74 | 50 |

### Example 1. Expression of Single Chain Trimer-Fc Fusion Protein (SCT-A2-Fc)

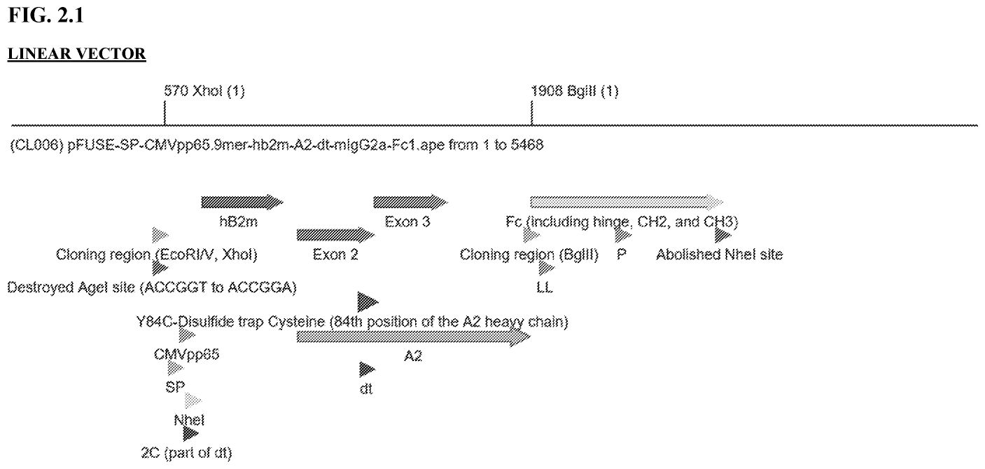

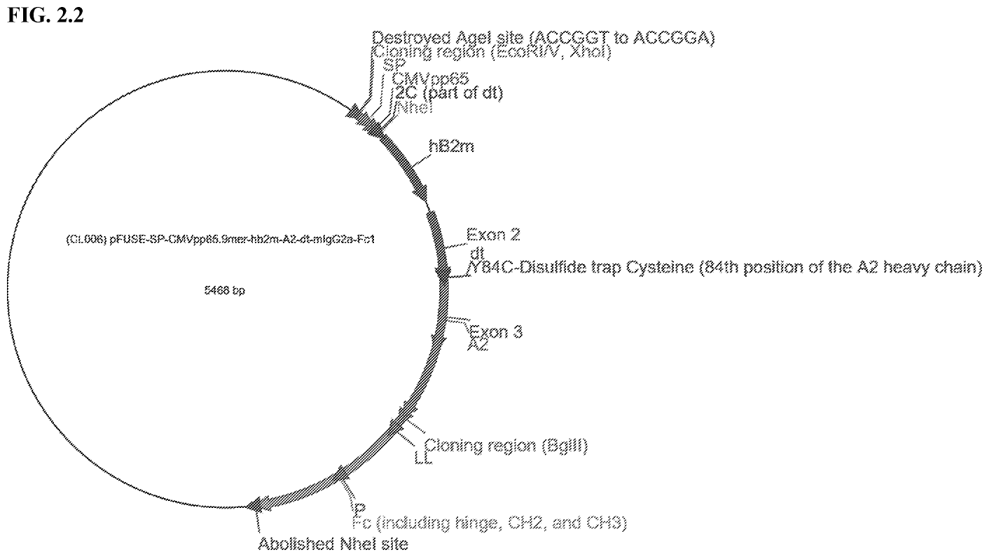

Using sequences provided in Table 10, DNA sequence encoding the CMVpp65 peptide (10mer) and the C-terminal end of the first linker was synthesized at IDT-DNA and cloned into vector WU1080 (see below) between the AgeI and NheI sites. Then the DNA sequence encoding the signal peptide (SP)-CMVpp65-human beta2 microglobulin-HLA-A2 (see Table 10) was amplified by PCR and cloned into the pFUSE-mIgG2a-Fc1 vector (Immunogen) between XhoI and BglII sites. Subsequently the construct was revised by replacing the DNA sequence between the AgeI site in the cloning region and the NheI site with synthesized sequence (IDT-DNA) encoding the signal peptide, a 9mer CMVpp65 peptide, and the C-terminal end of the first linker with a cysteine at position 2. This construct is the named “CL006” and is shown in linear form in FIG. 2.1 and circular form in FIG. 2.2 (SEQ ID NO: 69).

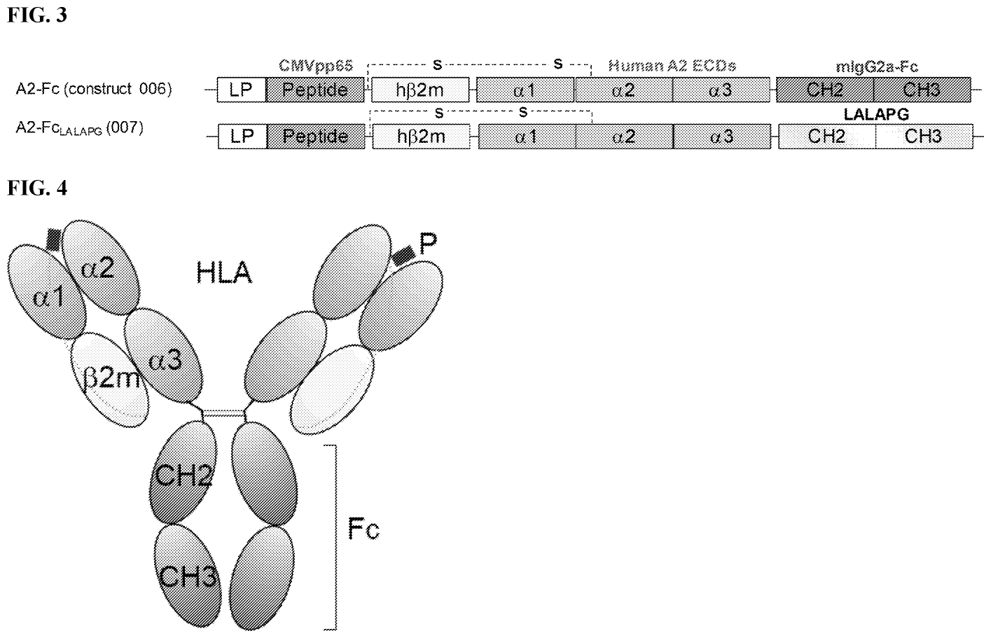

The SCT A2 protein, consisting of a peptide (e.g., G280-9V in the WU1080 plasmid), beta2-microglobulin, and HLA-A2, was created in according to previously described methods.^{43,44} The SCT A2-Fc fusion protein (#006, SEQ ID NO: 45) containing a CMVpp65 peptide (9mer) was designed as shown in FIG. 3 upper panel, and expressed it in the expi293 cell line. We also introduced point mutations to the Fc fragment to generate the control molecule A2-Fc_{LALAPG} (#007, SEQ ID NO: 46, FIG. 3, lower panel), which lacked all of the effector functions of Fc. The monomers SCT A2-Fc and SCT-Fc_{LALAPG} were predicted to dimerize and form an antibody-like structure (FIG. 4), except that both antigen-binding fragments (Fab) were replaced by extracellular domains (ECD) of A2. The CMVpp65 peptide was trapped in place by a disulfide bond.

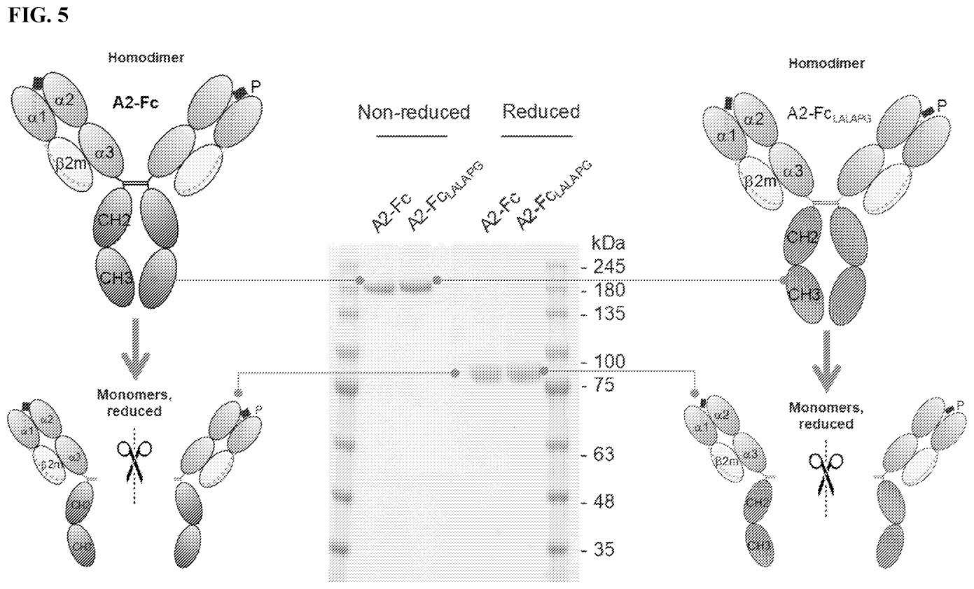

We successfully purified over 1 mg of each protein from 100 mL of culture by protein A chromatography. The expected sizes of the dimers (non-reduced) and monomers (reduced) were observed with the sodium dodecylsulfate polyacrylamide gel electrophoresis (SDS-PAGE) (FIG. 5).

### Example 2. A2-Fc Specifically Bind to MA2.1 Hybridoma Cells

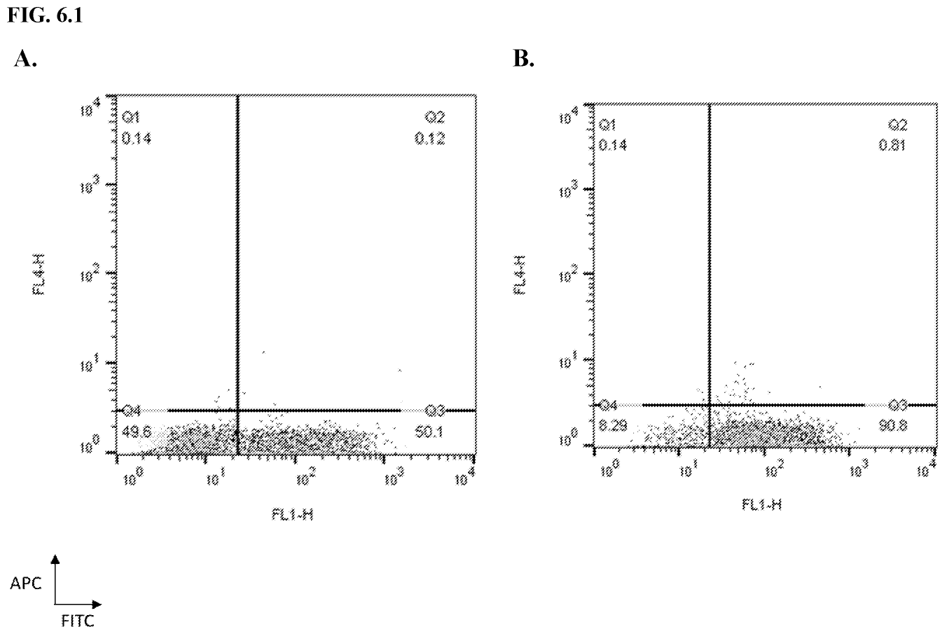

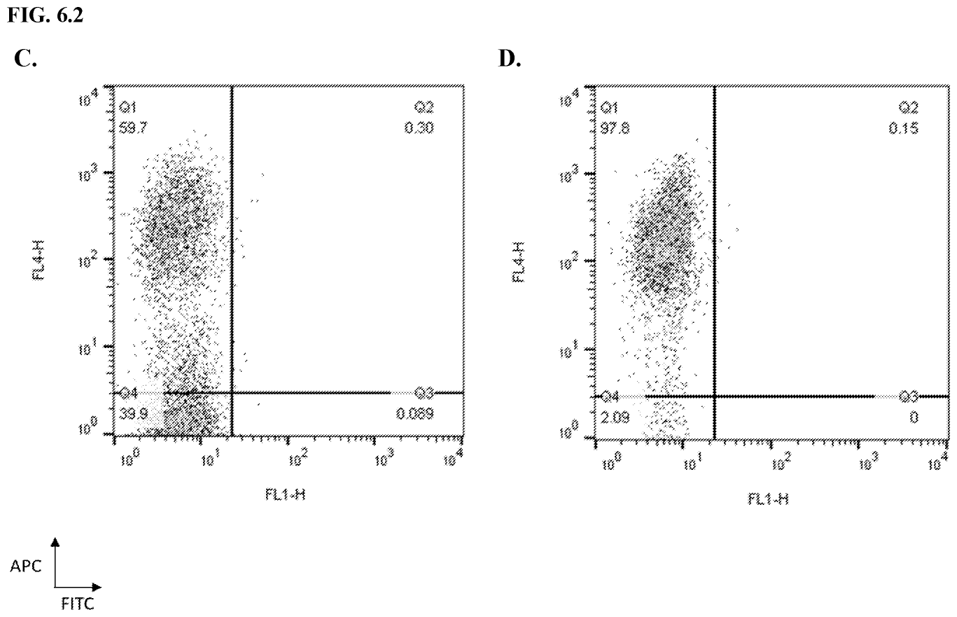

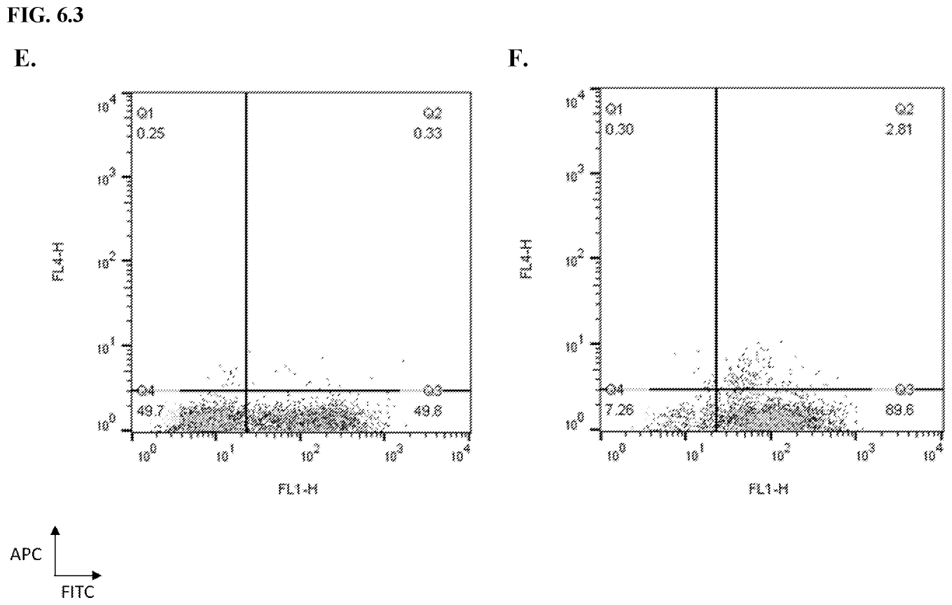

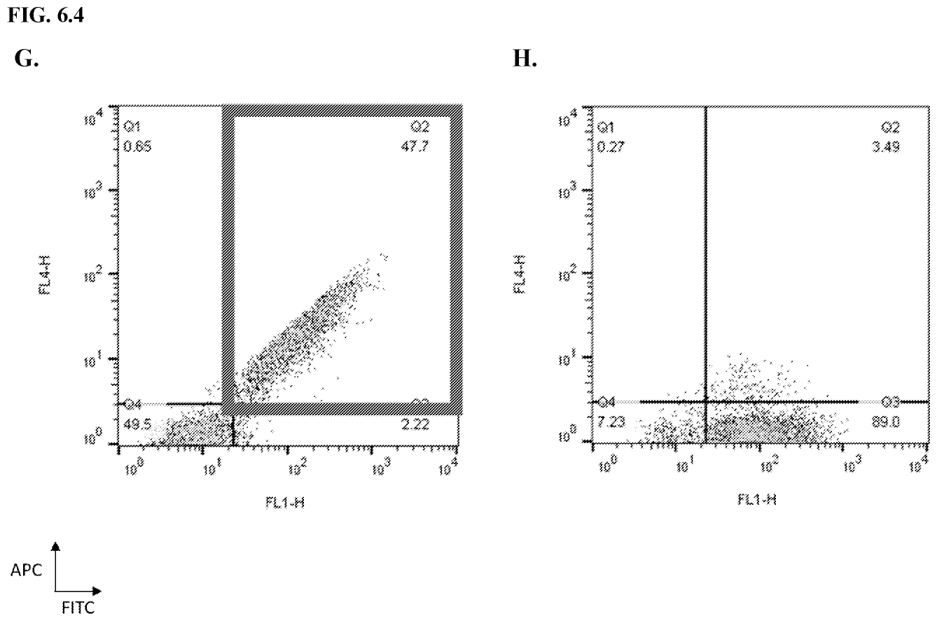

To test the specificity of the binding of the generated fusion proteins A2-Fc to hybridoma cells expressing anti-A2-IgG1 isotype B cell receptors (BCR), a flow cytometry experiment was performed. Specifically, hybridoma cell lines that express anti-A2 IgG1 isototype antibodies or anti-B7 IgG1 isotype antibodies (MA2.1 and BB7.1, respectively) were treated with A2-Fc and stained using anti-mIgG1 antibodies tagged with fluorescein (FITC) or anti-mIgG2a antibodies tagged with allophycocyanin (APC) as indicated in Table 12 below. Levels of A2-Fc (IgG2-type) binding to the B cell receptors (IgG1-type) expressed on these cells were determined by measuring the levels of APC fluorescence relative to FITC fluorescence. FIG. 6.1, FIG. 6.2, FIG. 6.3, and FIG. 6.4 show representative cytometry plots showing APC fluorescence (y-axis) relative to FITC fluorescence (x-axis) for MA2.1 cells (panels A, C, E, and G) or BB7.1 cells (panels B, D, F, and H) that were either untreated (panels A-F) or treated (panels G-H) with an anti-HLA-A2 antibody. MA2.1 cells (FIG. 6.4, panel G), but not BB7.1 cells (FIG. 6.4, panel H) showed an increase in APC fluorescence indicating that A2-Fc selectively bound to anti-A2 antibody generating cells and not anti-B7 antibody generating cells.

TABLE 12

| | Cell line | 006 treated | Staining |
|---|---|---|---|
| A | MA2.1 | − | Anti-mIgG1 -FITC |
| B | BB7.1 | − | Anti-mIgG1 -FITC |
| C | MA2.1 | − | Anti-mIgG1 -APC |
| D | BB7.1 | − | Anti-mIgG1 -APC |
| E | MA2.1 | − | Anti-mIgG1 -FITC |
| | | | Anti-mIgG2a-APC |
| F | BB7.1 | − | Anti-mIgG1 -FITC |
| | | | Anti-mIgG2a-APC |
| G | MA2.1 | + | Anti-mIgG1 -FITC |
| | | | Anti-mIgG2a-APC |
| H | BB7.1 | + | Anti-mIgG1 -FITC |
| | | | Anti-mIgG2a-APC |

### Example 3. A2-Fc, but not A2-Fc_{LALAPG} Causes Killing of MA2.1 Cells Via Complement Mediated Cytotoxicity Effect

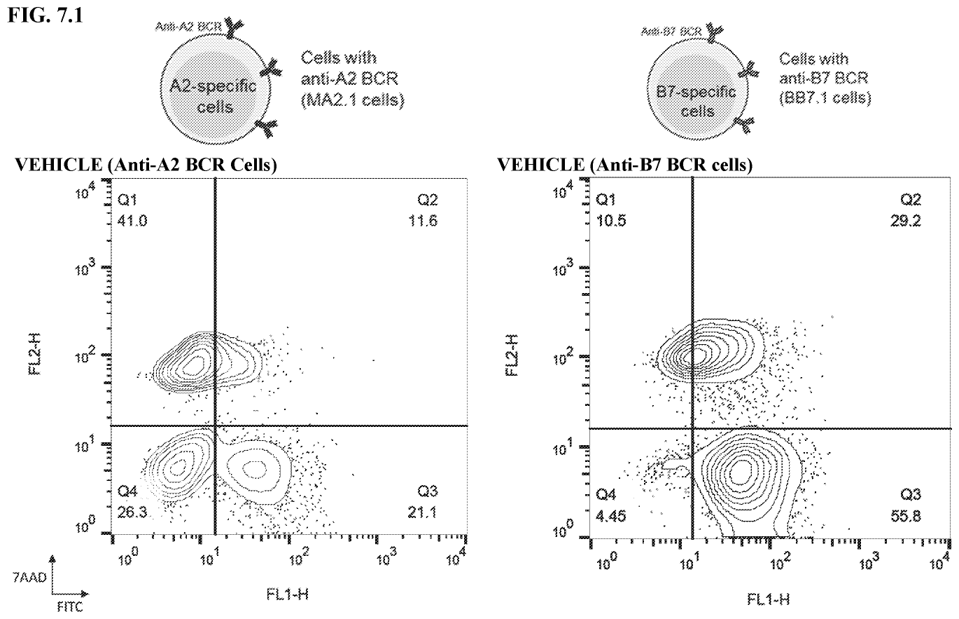

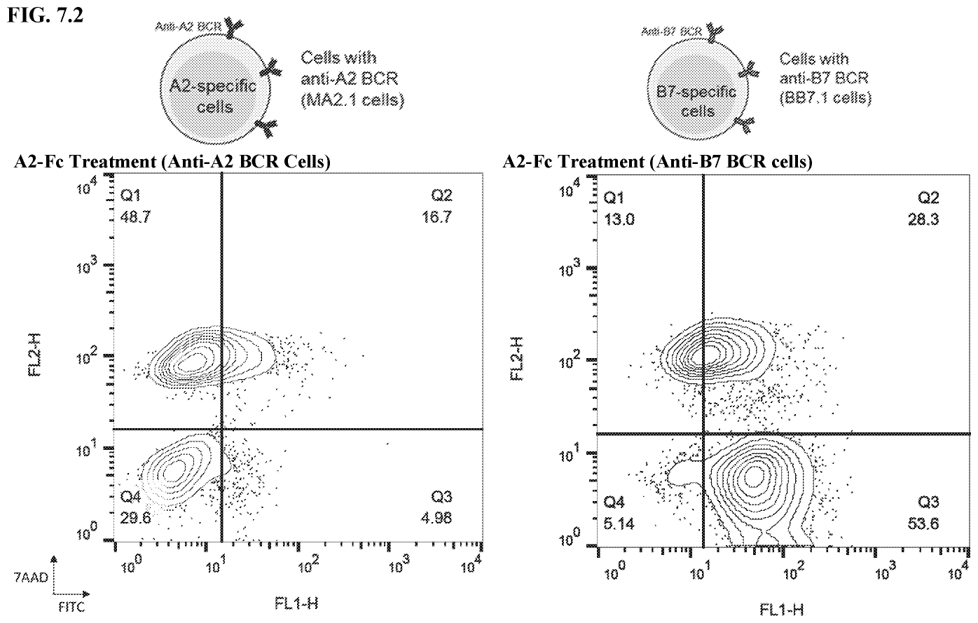

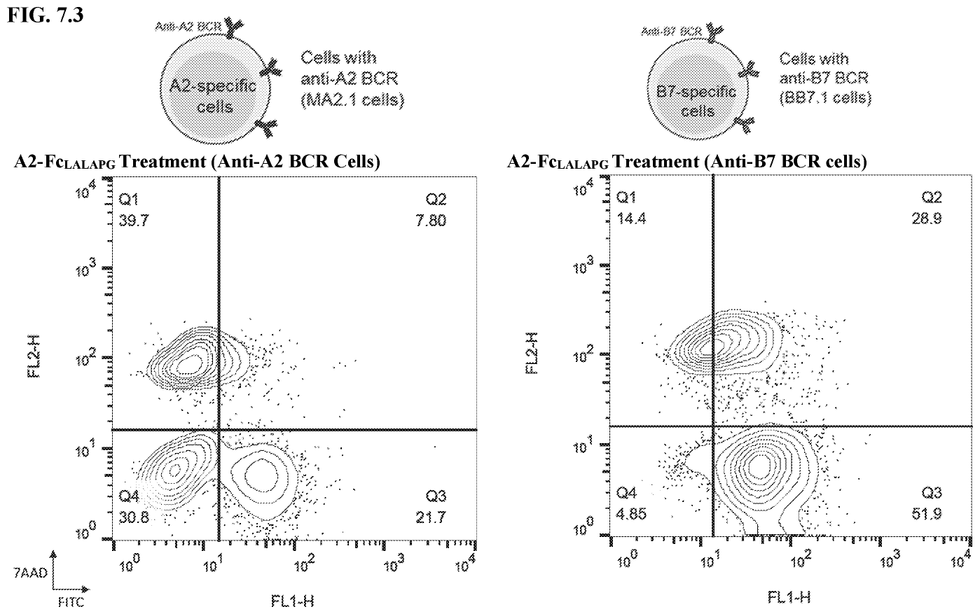

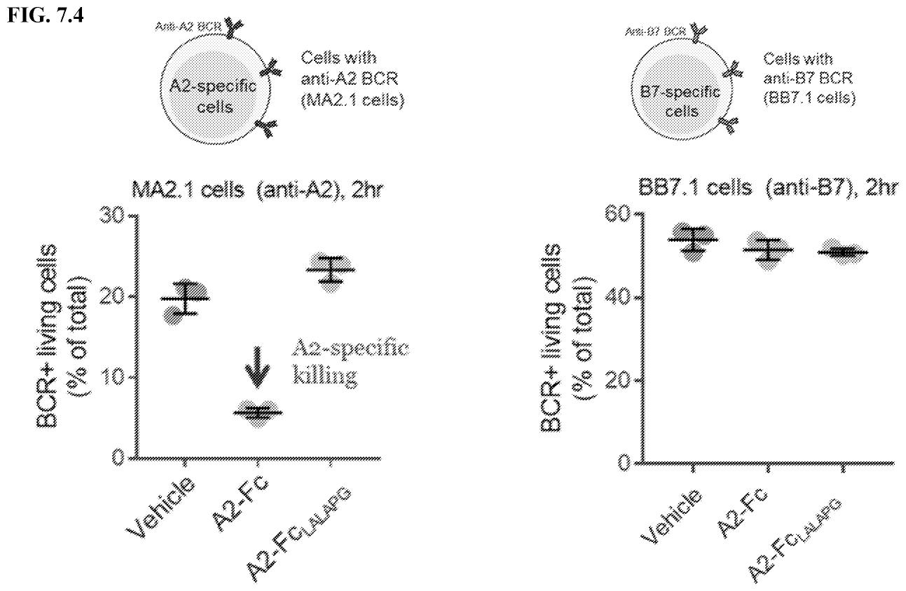

A2-Fc can bind to MA2.1 hybridoma cells expressing anti-A2 B cell receptors (BCR) but not BB7.1 hybridoma cells expressing anti-B7 BCR (see Example 2 and FIG. 6.4). To determine whether A2-Fc could successfully reduce levels of MA2.1 cells and to assess whether it could do so using a complement mediated cytotoxicity, a control fusion protein (A2-Fc_{LALAPG}) was generated (see Example 1). This fusion protein contained three mutations in the Fc portion of the protein (L19A, L20A, and P113G, e.g., SEQ ID NO 43 compared to SEQ ID NO: 42) that render it incapable of initiating the complement cascade. MA2.1 (anti-A2-BCR+) and BB7.1 (anti-B7 BCR+) hybridoma cells (ATCC, Manassas, Va.) were treated with a vehicle, the A2-Fc (006) or A2-Fc_{LALAPG} (007) followed by incubation with rabbit complement (One Lambda, Canoga Park, Calif.)) for two hours at room temperature. Cells were stained with 7-AAD to label dying cells and FITC-conjugated anti-mouse IgG1 to label BCR+ cells. FIG. 7.1, FIG. 7.2 and FIG. 7.3 show representative flow cytometry plots indicating 7AAD fluorescence (y-axis) relative to FITC fluorescence (x-axis) for each of the conditions indicated in Table 13 below. Summary plots depicting the total number of BCR+ cells remaining after treatment are shown in FIG. 7.4. Notably, only MA2.1 cells treated with the 006 fusion protein (A2-Fc) and not BB7.1 cells or MA2.1 cells treated with 007 (A2-Fc_{LALAPG}) were depleted following this experiment (FIG. 7.2 and FIG. 7.3). This data indicates that A2-Fc can selectively eliminate A2 specific cells via complement-dependent cytotoxicity.

TABLE 13

| | Cell | | |
|---|---|---|---|
| Figure Panel | line | Treatment | Staining |
| FIG. 7.1, left | MA2.1 | Vehicle, rabbit c 2 hr | 7AAD + Anti-mIgG1-FITC |
| FIG. 7.1, right | BB7.1 | Vehicle, rabbit c 2 hr | 7AAD + Anti-mIgG1-FITC |
| FIG. 7.2, left | MA2.1 | 006, rabbit c 2 hr | 7AAD + Anti-mIgG1-FITC |
| FIG. 7.2, right | BB7.1 | 006, rabbit c 2 hr | 7AAD + Anti-mIgG1-FITC |
| FIG. 7.3, left | MA2.1 | 007, rabbit c 2 hr | 7AAD + Anti-mIgG1-FITC |
| FIG. 7.3, right | BB7.1 | 007, rabbit c 2 hr | 7AAD + Anti-mIgG1-FITC |

### Example 4. Generation of HLA-Fc Proteins of Various Antigen and Peptide Specificities In Vitro

HLA are highly diverse and immunogenic. For the HLA-Fc protein to enable donor-specific immune suppression, the protein must be personalized to match the donor antigen that a patient is rejecting. To this end, additional HL-Fc fusion proteins were generated using antigen peptides specific for HLA-B complexes (specifically, HLA-B7). A diagram of the different fusion proteins generated is shown in FIG. 8. Specifically, the HLA-Fc proteins were generated using a similar approach as described in Example 1, except that the B7-specific SCT sequence is synthesized by IDT-DNA and in lieu of the CMVpp65 peptide (SEQ ID NO:1), three different peptides having different affinities for the B7 complex were used (see Table 14 below). In addition, a fourth B7 HLA construct was prepared (#010) that contained the original CMVpp65 sequence (a low affinity peptide for the B7 HLA chain, SEQ ID NO:1).

TABLE 14

| | Amino Acid | |
|---|---|---|
| | Sequence | Nucleic Acid Sequence |
| Name | (SEQ ID NO) | (SEQ ID NO) |
| Vaccinia | LPCQLMYAL | CTGCCCTGCCAGCTGATGTACG |
| virus: | (SEQ ID NO: | CCCTG (SEQ ID NO: 59) |
| L-9mer (#018) | 2) | |
| Dengue virus: | GPMKLVMAF | GGCCCCATGAAGCTGGTGATGG |
| G-9mer (#019) | (SEQ ID NO: | CCTTC (SEQ ID NO: 60) |
| | 3) | |
| Dengue virus: | HPGFTILAL | CACCCCGGCTTCACCATCCTGG |
| H-9mer (#020) | (SEQ ID NO: | CCCTG (SEQ ID NO: 61) |
| | 4) | |

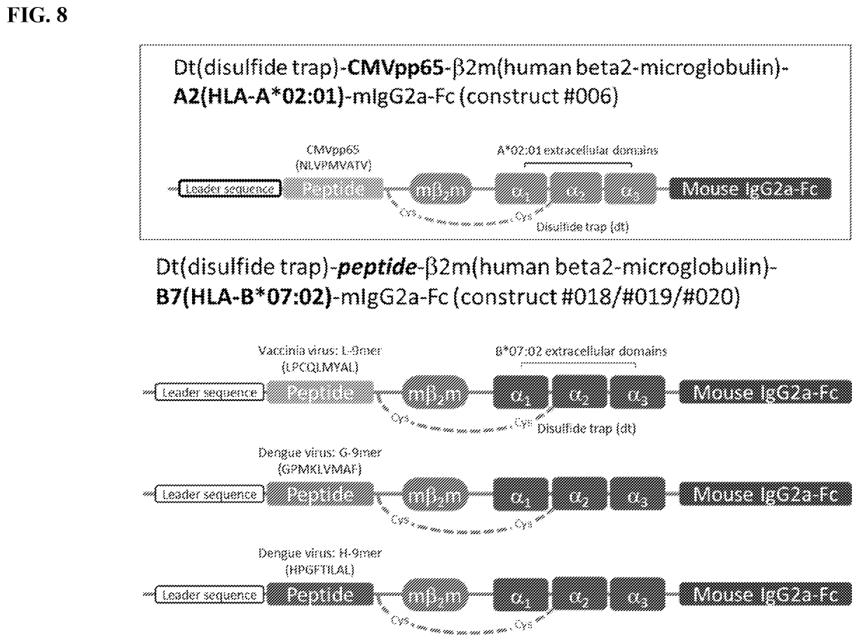

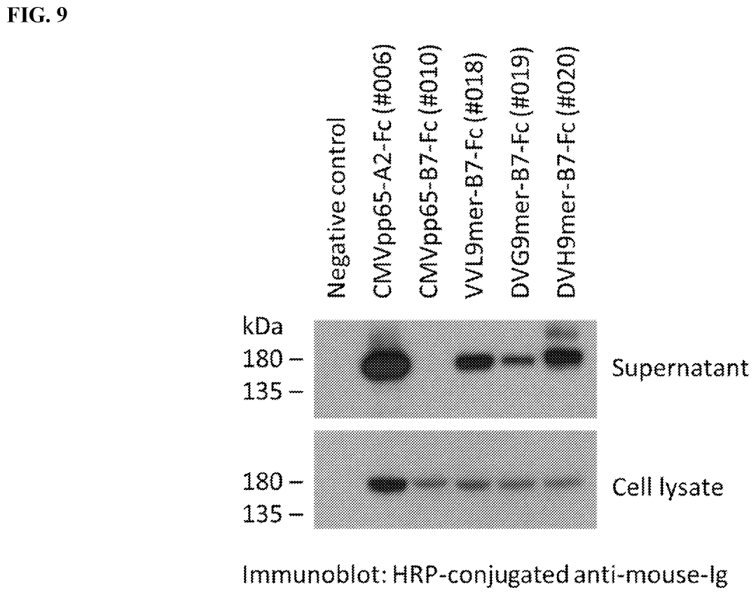

Each construct was transiently expressed in expi293 cells for 24 hours. Cell lysates and supernatants were analyzed by SDS-PAGE followed by western blot using HRP-conjugated anti-mouse-Ig. FIG. 9 shows a representative immunoblot of the expressed fusion proteins in the supernatant and cell lystate for each construct. Notably, B-7 constructs (#018, #019, and #020) containing peptides having a high affinity for HLA-B-7 were successfully expressed and detected in the supernatant, but the B-7 construct containing a low affinity peptide (e.g., CMVpp65) was not. This suggests that successful production of B7-Fc will be peptide-dependent since B7-Fc can only be expressed and released into the supernatant if coupled with a peptide of high affinity with HLA-B7.

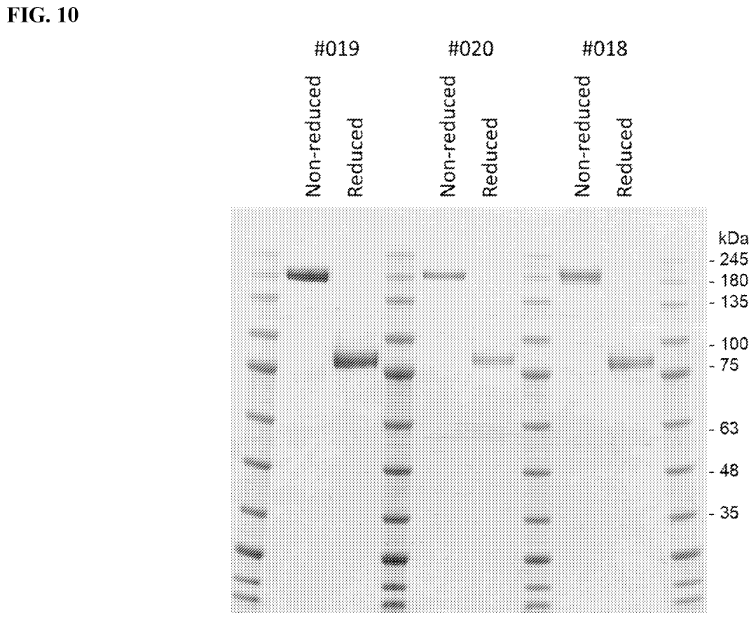

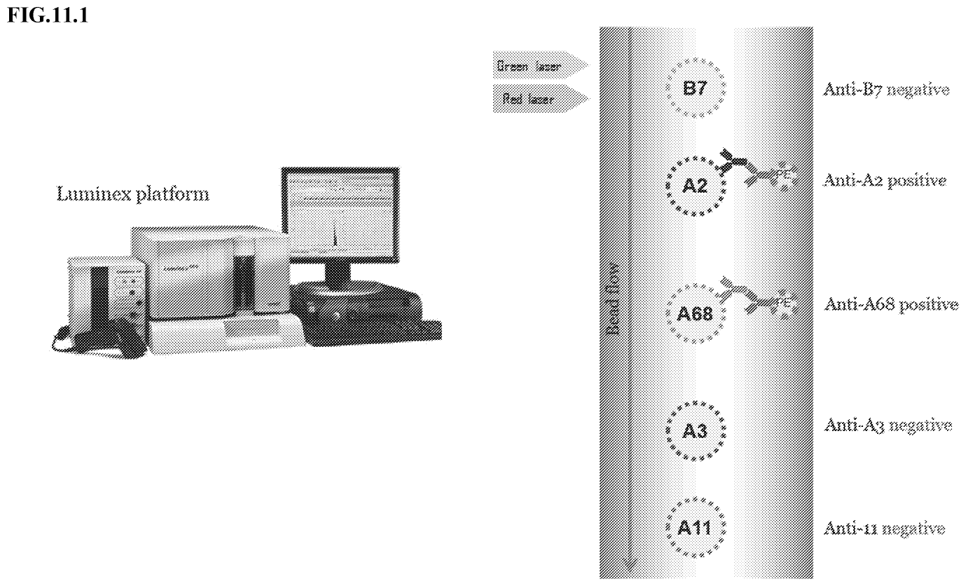

To test whether the B7-Fc constructs could form antibody-like homodimers, constructs were transiently transfected into 100 mL of expi293a cells (1 million cells/mL; half the standard transfection) and supernatant harvested (175 mL, cell count ˜2 million cells per mL) on day 5. Standard protein A chromatography was performed using a binding buffer (2×PBS pH=8) and elution in fraction #1-3 (1 mL each) Na-citrate at pH=5. A representative sodium dodecylsulfate polyacrylamide gel electrophoresis image is shown in FIG. 10 showing that each of the tested constructs could form antibody-like homodimers.

### Example 5. Using HLA-B7-Fc Fusion Proteins to Deplete B7 Selective Hybridomas In Vitro

Using the methods described in Example 3 above, the HLA-B7-Fc successfully expressed and characterized in Example 4, will be tested to determine their ability to trigger complement dependent depletion of B7 selective hybridoma cells in vitro. It is expected that HLA B7-Fc constructs showing robust expression and secretion into the supernatant (e.g., #018, #019, #020) will be effective in selectively decreasing anti-B7 expressing B-cells.

### Example 6. Using A2-Fc to Decrease the Production of Antibodies to HLA-A2 in a Murine Alloimmunization Model

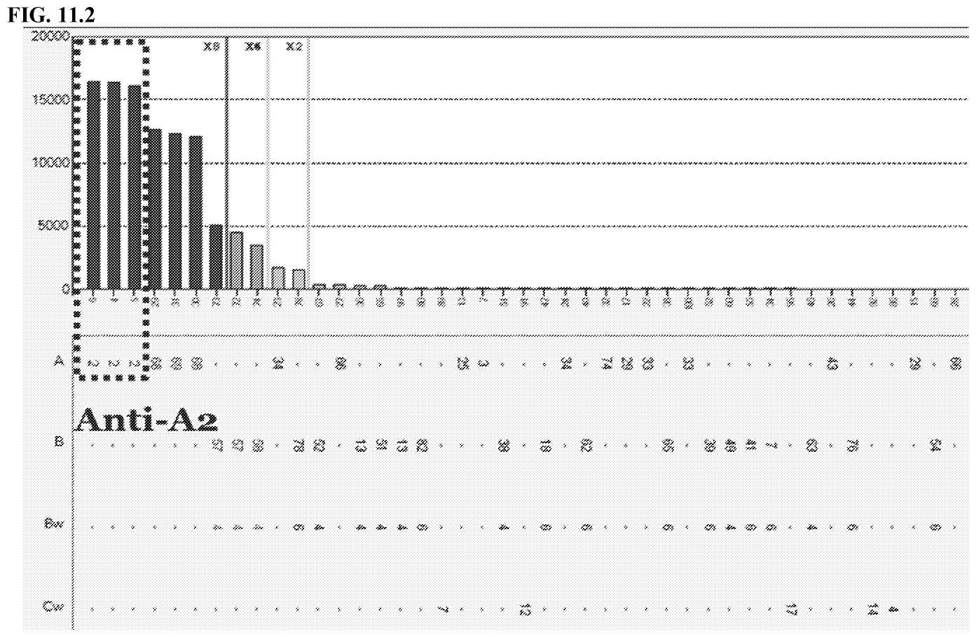

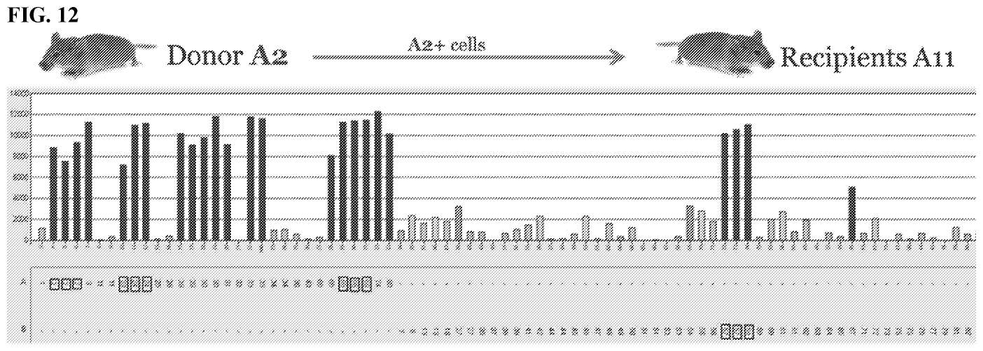

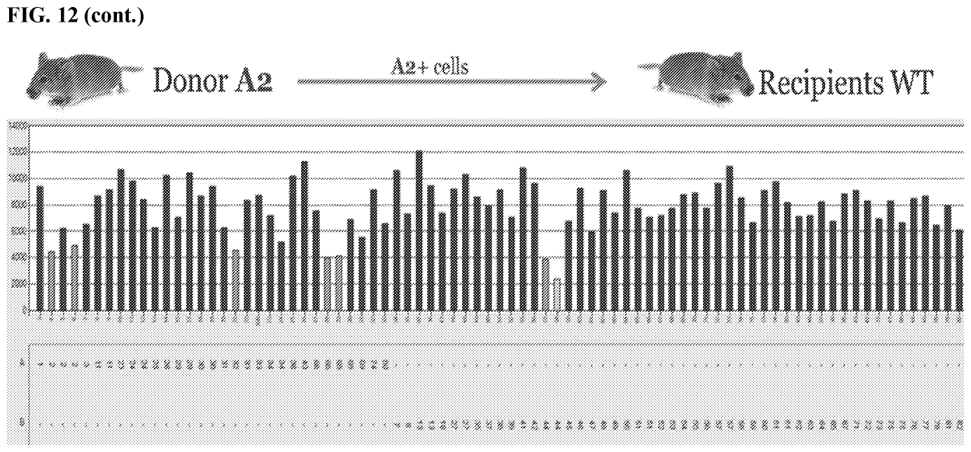

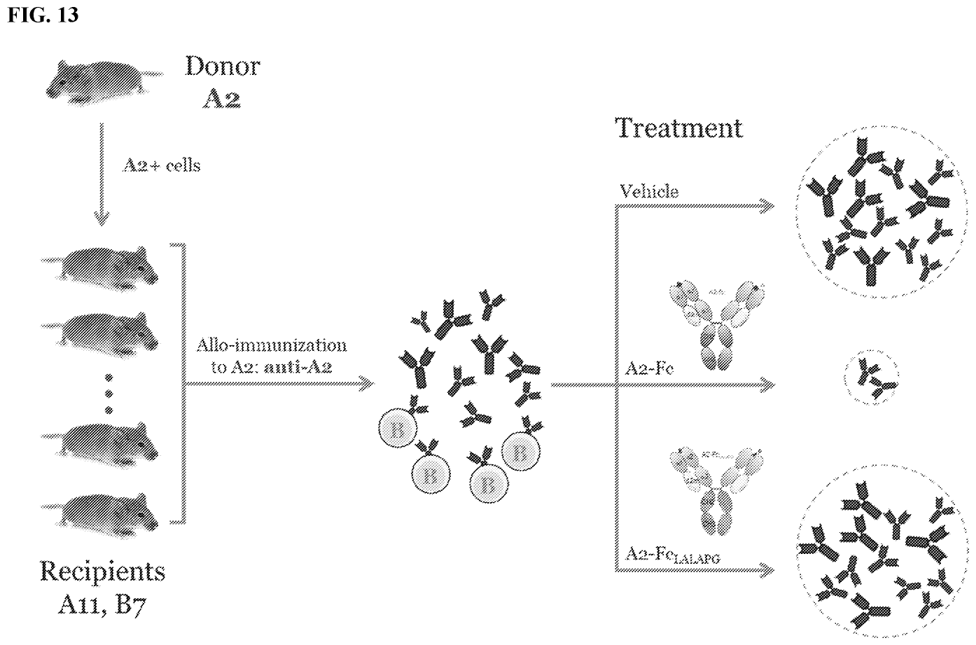

Overall strategy. Using a murine A2 alloimmunization model, we will test the hypothesis that HLA-A2-Fc can reduce the level of anti-A2 in vivo. C57BL/6 mice (“WT mice”) have been successfully immunized with skin grafts from transgenic C57BL.Tg/A2.1 mice.⁴⁹ We have created a model that differs in two aspects. First, instead of skin grafting, we induced the immune response by intraperitoneal injection of A2+ splenocytes into recipient mice. Second, we used CB6F1.Tg/A*11:01 mice (“A11 mice”)⁵⁰ instead of WT mice as the recipient and CB6F1.Tg/A*02:01 mice (“A2 mice”)⁵¹ as the donor to better mimic the transplant immunology in humans. FIG. 11.1 and FIG. 11.2 depict a LUMINEX assay used to measure various anti-HLA antibodies in mice following immunization with A2 specific splenocytes. Specifically, samples were mixed with beads coated with various HLA proteins and levels of anti-HLA antibodies correlated with mean fluorescence intensity (MFI) detected for each bead population. FIG. 11.2 shows how anti-A2 antibodies can be specifically identified based on the MFI signature of the sample. FIG. 12 shows the MFI of a population of antibodies in an A11 mouse immunized with A2 cells (upper panel) or a wildtype (WT) mouse immunized with A2 cells (lower panel). WT mice challenged with the A2+ splenocytes were found to launch a broad humoral response that cross-reacts with most class I HLA-A, -B, and -C antigens (FIG. 12, lower panel). A11 mice instead only responded to a smaller subset of epitopes in A2 that are not shared by A11 (FIG. 12, upper panel). The specificity of these A11 mice to the A2 epitope will ensure that the A2-Fc fusion protein, once delivered into these mice should attenuate the anti-A2 level by targeting A2-specific B cells via CDC or ADCC (FIG. 13). A detailed methodology and experimental protocol for this experiment is described below.

Methodology 1) Alloimmunization model: Splenocytes will be harvested from adult A2 mice, and 5 million cells will be transferred to each A11 mouse (n=3) via intraperitoneal injection. Approximately 200 μL peripheral blood will be collected via retro-orbital bleed before immunization and at 1 and 4 weeks post-immunization. Plasma will be stored at −20° C. and batch tested by the SAB assay (see above) to confirm the alloimmunization. 2) Treatment & blood sampling: Immunized mice will be divided into three treatment groups (n=5 per group): A2-Fc, A2-Fc_{LALAPG} (Control-1), and A11-Fc (Control-2). Treatments will be administered at 2 weeks and 6 weeks post-immunization by intraperitoneal injection at an empirical dose of 30 mg/kg, and blood will be sampled before immunization and at 1, 4, 8, and 12 weeks post-immunization for antibody testing. 3) Anti-A2 measurement by SAB assay & FCXM. Stored plasma will be tested by the standard SAB assay in the BJH HLA laboratory,⁵² except that PE-conjugated anti-mouse IgG1 and anti-mouse total Ig will be used as the secondary antibody. Serially diluted plasma at titers of 1, 4, and 16 will be crossmatched against A2+ splenocytes using standard FCXM technique in the BJH HLA laboratory,⁵³ except that FITC-conjugated anti-mouse IgG1 (Fab) will be used as the secondary antibody. Non-sensitized plasma will be used as controls to provide baseline median channel numbers, against which the median channel shift (MCS) will be calculated. 5) Quantification of A2-specific B cells. A2-specific B cells in the peripheral blood and spleen at 10 weeks after the first treatment will be stained with FITC-conjugated A2-tetramer followed by flow cytometry. We will also stain CD220 (total B cells), CD5 (B-1a cells), and CD3 (total T cells) to quantify the total T and B cell populations.

Analysis & anticipated results. The mean fluorescence intensity (MFI) values from the SAB assay and MCS from FCXM will be reported as mean±SEM per group for each time point. The number of A2-specific B cells as a percentage of the B220+/CD5-B cells will be reported as mean±SEM % per group. Comparison among groups will be performed by Kruskal-Wallis one-way analysis of variance (non-parametric ANOVA), followed by Mann-Whitney U test for two-group comparison. We have already found successful alloimmunization of A11 mice by A2+ splenocytes (FIG. 12). We also anticipate a significant decrease in MFI and MCS values in A2-Fc treated mice compared to mice treated by controls post-treatment as measured by the SAB assay and FCXM. The percentage of A2-specific B cells should also decrease significantly after A2-Fc treatment.

In general this example will show that the HLA-Fc fusion protein described herein can to provide a personalized solution to enable selective B cell depletion for desensitization and AMR treatment. It may also offer a universal strategy to remove unwanted alloantibodies and pathological autoantibodies.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description and shown in the accompanying drawings shall be interpreted as illustrative and not in a limiting sense.

### REFERENCES

1. Baxter-Lowe L A, Kucheryavaya A, Tyan D, Reinsmoen N. CPRA for allocation of kidneys in the US: More candidates ≥98% CPRA, lower positive crossmatch rates and improved transplant rates for sensitized patients. Human Immunology 2016; 77:395-402.
2. Orandi B J, Luo X, Massie A B, et al. Survival Benefit with Kidney Transplants from HLA-Incompatible Live Donors. N Engl J Med 2016; 374:940-50.
3. Hannan A, Nogueira J, Kukuruga D, et al. Positive cross-match living donor kidney transplantation: longer-term outcomes. Am J Transplant 2009; 9:536-42.
4. Bentall A, Cornell L D, Gloor J M, et al. Five-year outcomes in living donor kidney transplants with a positive crossmatch. Am J Transplant 2013; 13:76-85.
5. Gloor J M, Winters J L, Cornell L D, et al. Baseline donor-specific antibody levels and outcomes in positive crossmatch kidney transplantation. Am J Transplant 2010; 10:582-9.
6. Orandi B J, Garonzik-Wang J M, Massie A B, et al. Quantifying the risk of incompatible kidney transplantation: a multicenter study. Am J Transplant 2014; 14:1573-80.
7. Mohan S, Palanisamy A, Tsapepas D, et al. Donor-specific antibodies adversely affect kidney allograft outcomes. J Am Soc Nephrol 2012; 23:2061-71.
8. Djamali A, Kaufman D B, Ellis T M, Zhong W, Matas A, Samaniego M. Diagnosis and management of antibody-mediated rejection: current status and novel approaches. Am J Transplant 2014; 14:255-71.
9. Sellares J, de Freitas D G, Mengel M, et al. Understanding the causes of kidney transplant failure: the dominant role of antibody-mediated rejection and nonadherence. Am J Transplant 2012; 12:388-99.
10. Wiebe C, Gibson I W, Blydt-Hansen T D, et al. Evolution and clinical pathologic correlations of de novo donor-specific HLA antibody post kidney transplant. Am J Transplant 2012; 12:1157-67.
11. El-Zoghby Z M, Stegall M D, Lager D J, et al. Identifying specific causes of kidney allograft loss. Am J Transplant 2009; 9:527-35.
12. Tambur A R, Campbell P, Claas F H, et al. Sensitization in Transplantation: Assessment of Risk (STAR) 2017 Working Group Meeting Report. Am J Transplant 2018.
13. Spellman S, Bray R, Rosen-Bronson S, et al. The detection of donor-directed, HLA-specific alloantibodies in recipients of unrelated hematopoietic cell transplantation is predictive of graft failure. Blood 2010; 115:2704-8.
14. Ciurea S O, Thall P F, Wang X, et al. Donor-specific anti-HLA Abs and graft failure in matched unrelated donor hematopoietic stem cell transplantation. Blood 2011; 118:5957-64.
15. Witt C A, Gaut J P, Yusen R D, et al. Acute antibody-mediated rejection after lung transplantation. J Heart Lung Transplant 2013; 32:1034-40.
16. Kulkarni H S, Bemiss B C, Hachem R R. Antibody-mediated Rejection in Lung Transplantation. Curr Transplant Rep 2015; 2:316-23.
17. Aguilar P R, Carpenter D, Ritter J, et al. The role of C4d deposition in the diagnosis of antibody-mediated rejection after lung transplantation. Am J Transplant 2018; 18:936-44.
18. Mangiola M, Marrari M, Feingold B, Zeevi A. Significance of Anti-HLA Antibodies on Adult and Pediatric Heart Allograft Outcomes. Front Immunol 2017; 8:4.
19. Tran A, Fixler D, Huang R, Meza T, Lacelle C, Das B B. Donor-specific HLA alloantibodies: Impact on cardiac allograft vasculopathy, rejection, and survival after pediatric heart transplantation. J Heart Lung Transplant 2016; 35:87-91.
20. Irving C A, Carter V, Gennery A R, et al. Effect of persistent versus transient donor-specific HLA antibodies on graft outcomes in pediatric cardiac transplantation. J Heart Lung Transplant 2015; 34:1310-7.
21. O'Leary J G, Kaneku H, Banuelos N, Jennings L W, Klintmalm G B, Terasaki P I. Impact of IgG3 subclass and C1q-fixing donor-specific HLA alloantibodies on rejection and survival in liver transplantation. Am J Transplant 2015; 15:1003-13.
22. O'Leary J G, Demetris A J, Friedman L S, et al. The role of donor-specific HLA alloantibodies in liver transplantation. Am J Transplant 2014; 14:779-87.
23. O'Leary J G, Kaneku H, Demetris A J, et al. Antibody-mediated rejection as a contributor to previously unexplained early liver allograft loss. Liver Transpl 2014; 20:218-27.
24. O'Leary J G, Kaneku H, Jennings L W, et al. Preformed class II donor-specific antibodies are associated with an increased risk of early rejection after liver transplantation. Liver Transpl 2013; 19:973-80.
25. O'Leary J G, Kaneku H, Susskind B M, et al. High mean fluorescence intensity donor-specific anti-HLA antibodies associated with chronic rejection Postliver transplant. Am J Transplant 2011; 11:1868-76.
26. Jordan S C, Lorant T, Choi J, et al. IgG Endopeptidase in Highly Sensitized Patients Undergoing Transplantation. New England Journal of Medicine 2017; 377:442-53.
27. Sautenet B, Blancho G, Buchler M, et al. One-year Results of the Effects of Rituximab on Acute Antibody-Mediated Rejection in Renal Transplantation: RITUX ERAH, a Multicenter Double-blind Randomized Placebo-controlled Trial. Transplantation 2016; 100:391-9.
28. Ejaz N S, Shields A R, Alloway R R, et al. Randomized controlled pilot study of B cell-targeted induction therapy in HLA sensitized kidney transplant recipients. Am J Transplant 2013; 13:3142-54.
29. Cheungpasitporn W, Thongprayoon C, Edmonds P J, Bruminhent J, Tangdhanakanond K. The effectiveness and safety of rituximab as induction therapy in ABO-compatible non-sensitized renal transplantation: a systematic review and meta-analysis of randomized controlled trials. Ren Fail 2015; 37:1522-6.
30. Eskandary F, Regele H, Baumann L, et al. A Randomized Trial of Bortezomib in Late Antibody-Mediated Kidney Transplant Rejection. J Am Soc Nephrol 2018; 29:591-605.
31. Moreno Gonzales M A, Gandhi M J, Schinstock C A, et al. 32 Doses of Bortezomib for Desensitization Is Not Well Tolerated and Is Associated With Only Modest Reductions in Anti-HLA Antibody. Transplantation 2017; 101:1222-7.
32. Kulkarni S, Kirkiles-Smith N C, Deng Y H, et al. Eculizumab Therapy for Chronic Antibody-Mediated Injury in Kidney Transplant Recipients: A Pilot Randomized Controlled Trial. American Journal of Transplantation 2017; 17:682-91.
33. Cornell L D, Schinstock C A, Gandhi M J, Kremers W K, Stegall M D. Positive crossmatch kidney transplant recipients treated with eculizumab: outcomes beyond 1 year. Am J Transplant 2015; 15:1293-302.
34. Vacha M, Chery G, Hulbert A, et al. Antibody depletion strategy for the treatment of suspected antibody-mediated rejection in lung transplant recipients: Does it work? Clin Transplant 2017; 31.
35. Patel J, Everly M, Chang D, Kittleson M, Reed E, Kobashigawa J. Reduction of alloantibodies via proteasome inhibition in cardiac transplantation. J Heart Lung Transplant 2011; 30:1320-6.
36. Trivin C, Tran A, Moulin B, et al. Infectious complications of a rituximab-based immunosuppressive regimen in patients with glomerular disease. Clin Kidney J 2017; 10:461-9.
37. Zachary A A, Lucas D P, Montgomery R A, Leffell M S. Rituximab prevents an anamnestic response in patients with cryptic sensitization to HLA. Transplantation 2013; 95:701-4.
38. Zachary A A, Kopchaliiska D, Montgomery R A, Leffell M S. HLA-specific B cells: I. A method for their detection, quantification, and isolation using HLA tetramers. Transplantation 2007; 83:982-8.
39. Liwski R S, Greenshields A L, Bray R A, Gebel H M. Becoming a chef in the human leukocyte antigen kitchen: interpretation and modification of human leukocyte antigen-antibody assays. Curr Opin Organ Transplant 2017; 22:407-14.
40. Elliott M J, Maini R N, Feldmann M, et al. Randomised double-blind comparison of chimeric monoclonal antibody to tumour necrosis factor alpha (cA2) versus placebo in rheumatoid arthritis. Lancet 1994; 344:1105-10.
41. Monaco C, Nanchahal J, Taylor P, Feldmann M. Anti-TNF therapy: past, present and future. Int Immunol 2015; 27:55-62.
42. Moreland L W, Baumgartner S W, Schiff M H, et al. Treatment of rheumatoid arthritis with a recombinant human tumor necrosis factor receptor (p75)-Fc fusion protein. N Engl J Med 1997; 337:141-7.
43. Hansen T, Yu Y Y, Fremont D H. Preparation of stable single-chain trimers engineered with peptide, beta2 microglobulin, and MHC heavy chain. Curr Protoc Immunol 2009; Chapter 17:Unit17.5.
44. Mulder A, Eijsink C, Kester M G, et al. Impact of peptides on the recognition of HLA class I molecules by human HLA antibodies. J Immunol 2005; 175:5950-7.
45. Gebel H M, Bray R A. HLA Antibody Detection With Solid Phase Assays: Great Expectations or Expectations Too Great? Am J Transplant 2014.
46. Chamow S M, Ryll T, Lowman H B, Farson D. Therapeutic Fc-Fusion Proteins2013.
47. Petersen T N, Brunak S, von Heijne G, Nielsen H. SignalP 4.0: discriminating signal peptides from transmembrane regions. Nat Methods 2011; 8:785-6.
48. von Heijne G. A new method for predicting signal sequence cleavage sites. Nucleic Acids Res 1986; 14:4683-90.
49. Wu G D, He Y, Chai N N, et al. Anti-CD20 antibody suppresses anti-HLA antibody formation in a HLA-A2 transgenic mouse model of sensitization. Transplant immunology 2008; 19:178-86.
50. Alexander J, Oseroff C, Sidney J, et al. Derivation of HLA-A11/Kb transgenic mice: functional CTL repertoire and recognition of human A11-restricted CTL epitopes. J Immunol 1997; 159:4753-61.
51. Vitiello A, Marchesini D, Furze J, Sherman L A, Chesnut R W. Analysis of the HLA-restricted influenza-specific cytotoxic T lymphocyte response in transgenic mice carrying a chimeric human-mouse class I major histocompatibility complex. J Exp Med 1991; 173:1007-15.
52. Liu C, Pang S, Phelan D, Brennan D C, Mohanakumar T. Quantitative Evaluation of the Impact of Ethylenediaminetetraacetic Acid Pretreatment on Single-Antigen Bead Assay. Transplant Direct 2017; 3:e194.
53. Liwski R S, Greenshields A L, Conrad D M, et al. Rapid optimized flow cytometric crossmatch (FCXM) assays: The Halifax and Halifaster protocols. Hum Immunol 2018; 79:28-38.
54. Xiang Z, Cutler A J, Brownlie R J, et al. Fc[gamma]RIIb controls bone marrow plasma cell persistence and apoptosis. Nat Immunol 2007; 8:419-29.

What is claimed is:

1. A fusion protein comprising a single chain trimer (SCT) and a fragment crystallizable (Fc) region of an antibody, wherein the SCT comprises an antigen peptide, a first flexible linker, a β2-microglobulin, a second flexible linker, and a MHC class I heavy chain.

2. The fusion protein of claim 1 wherein the first flexible linker and the second flexible linker each independently comprises at least 8, at least 10, at least 11, at least 12, at least 13, at least 14, at least 15, at least 16, at least 17, at least 18, at least 19 or at least 20 amino acid residues.

3. The fusion protein of claim 1 or 2 wherein the first flexible linker and the second flexible linker each independently comprises from 10 to 25 amino acid residues, or from 15 to 20 amino acid residues.

4. The fusion protein of any one of claims 1 to 3 wherein the first flexible linker and the second flexible linker each independently comprises about 10, about 11, about 12, about 13, about 14, about 15, about 16, about 17, about 18, about 19, or about 20 amino acid residues.

5. The fusion protein of any one of claims 1 to 4 wherein the first flexible linker and the second flexible linker each independently comprises 80% or more glycine, alanine and/or serine residues.

6. The fusion protein of any one of claims 1 to 5 wherein the first flexible linker comprises an amino acid sequence comprising greater than 80%, greater than 85%, or greater than 90% sequence identity to any one of SEQ ID NOs 31-34 and 37-41.

7. The fusion protein of any one of claims 1 to 6 wherein the first flexible linker comprises an amino acid sequence comprising any one of SEQ ID NOs: 31-34 and 37-41.

8. The fusion protein of any one of claims 1 to 7 wherein the second flexible linker comprises an amino acid sequence comprising greater than 80%, greater than 85%, or greater than 90% sequence identity to SEQ ID NO 35 or 36.

9. The fusion protein of any one of claims 1 to 8 wherein the second flexible linker comprises an amino acid sequence comprising SEQ ID NO: 35 or 36.

10. The fusion protein of any one of claims 1 to 9 wherein the antigen peptide comprises from 8 to 15, from 8 to 14, from 8 to 13, from 8 to 12, from 8 to 11, or from 8 to 10 amino acid residues.

11. The fusion protein of any one of claims 1 to 10 wherein the antigen peptide comprises an antigen peptide that can bind to the MHC class I heavy chain.

12. The fusion protein of any one of claims 1 to 11 wherein the antigen peptide comprises a human leukocyte antigen-A (HLA-A) restricted peptide, a HLA-B restricted peptide, a HLA-C restricted peptide, a HLA-E restricted peptide, a HLA-F restricted peptide, or a HLA-G restricted peptide.

13. The fusion protein of any one of claims 1 to 12 wherein the antigen peptide comprises a HLA-A restricted peptide or a HLA-B restricted peptide.

14. The fusion protein of any one of claims 1 to 13 wherein the antigen peptide comprises a HLA-A*02 restricted peptide, a HLA-A*11 restricted peptide, or a HLA-B*07 restricted peptide.

15. The fusion protein of any one of claims 1 to 14 wherein the antigen peptide comprises an amino acid sequence having greater than 80%, greater than 85%, or greater than 90% sequence identity to any one of SEQ ID NOs: 1-22.

16. The fusion protein of any one of claims 1 to 15 wherein the antigen peptide comprises any one of SEQ ID NOs: 1-22.

17. The fusion protein of any one of claims 1 to 16 wherein the antigen peptide comprises any one of SEQ ID NOs: 1-4.

18. The fusion protein of claim 1 wherein the antigen peptide comprises any one of SEQ ID NOs: 1-4.

19. The fusion protein of any one of claims 1 to 18 wherein the antigen peptide promotes stabilization and synthesis of the fusion protein.

20. The fusion protein of any one of claims 1 to 19 wherein the β2 microglobulin comprises a mammalian β2 microglobulin.

21. The fusion protein of any one of claims 1 to 20 wherein the β2 microglobulin comprises a human or murine β2 microglobulin.

22. The fusion protein of any one of claims 1 to 21 wherein the β2 microglobulin comprises an amino acid sequence having greater than 80%, greater than 85%, greater than 90%, or greater than 95% sequence identity to SEQ ID NO: 23 or SEQ ID NO: 24.

23. The fusion protein of any one of claims 1 to 22 wherein the β2 microglobulin comprises an amino acid sequence comprising SEQ ID NO: 23 or SEQ ID NO: 24.

24. The fusion protein of claim 1 wherein the β2-microglobulin comprises an amino acid sequence comprising SEQ ID NO: 23 or SEQ ID NO: 24.

25. The fusion protein of any one of claims 1 to 24 wherein the MHC class I heavy chain comprises a human or a murine MHC class I heavy chain.

26. The fusion protein of any one of claims 1 to 25 wherein the MHC class I heavy chain comprises a human leukocyte antigen (HLA) heavy chain.

27. The fusion protein of any one of claims 1 to 26 wherein the MHC class I heavy chain comprises a HLA-A, a HLA-B, a HLA-C, a HLA-E, a HLA-F, or a HLA-G heavy chain.

28. The fusion protein of any one of claims 1 to 27 wherein the MHC class I heavy chain comprises a HLA-A, a HLA-B, or a HLA-C heavy chain.

29. The fusion protein of any one of claims 1 to 28 wherein the MHC class I heavy chain comprises HLA-A*02, HLA-A*11, or HLA-B*07.

30. The fusion protein of any one of claims 1 to 29 wherein the MHC class I heavy chain comprises any one of SEQ ID NOs: 25-30.

31. The fusion protein of any one of claims 1 to 30 wherein a first residue in the first flexible linker and a second residue in the MHC class I heavy chain are linked by a covalent bond.

32. The fusion protein of claim 31 wherein the first residue is a first cysteine residue, the second residue is a second cysteine residue and the covalent bond comprises a disulfide bridge.

33. The fusion protein of claim 32 wherein the second cysteine residue is located from 1 to 100, from 10 to 100, from 20 to 100, from 30 to 100, from 40 to 100, from 50 to 100, from 55 to 100, from 60 to 100, from 60 to 90, from 65 to 90, from 70 to 90, or from 80 to 90 amino acid residues from the amino terminus of the MHC class I heavy chain.

34. The fusion protein of any one of claims 1 to 33 wherein the MHC class I heavy chain has an amino acid sequence having at least 90%, at least 95% or at least 99% sequence identity to any one of SEQ ID NO: 26, 28, and 30 comprising at least one amino acid substitution selected from the group consisting of Y84C, T80C, and A86C.

35. The fusion protein of any one of claims 1 to 34 wherein the MHC class I heavy chain has an amino acid sequence comprising any one of SEQ ID NOs: 25, 27, and 29.

36. The fusion protein of claim 1 wherein the MHC class I heavy chain comprises an amino acid sequence comprising any one of SEQ ID NOs: 25, 27 and 29.

37. The fusion protein of any one of claims 1 to 36 wherein the first flexible linker comprises an amino acid sequence comprising any one of SEQ ID NOs: 37-41.

38. The fusion protein of any one of claims 1 to 37 wherein the first flexible linker comprises an amino acid sequence comprising SEQ ID NO: 38.

39. The fusion protein of claim 1 wherein the first flexible linker comprises an amino acid sequence comprising SEQ ID NO: 38.

40. The fusion protein of any one of claims 1 to 39 wherein the Fc region comprises the Fc region of a human, murine, rabbit, or goat antibody.

41. The fusion protein of any one of claims 1 to 40 wherein the Fc region comprises the Fc region of a human or murine antibody.

42. The fusion protein of any one of claims 1 to 41 wherein the Fc region comprises an Fc segment of an IgG antibody.

43. The fusion protein of any one of claims 1 to 42 wherein the Fc region is capable of initiating complement.

44. The fusion protein of any one of claims 1 to 43 wherein the Fc region comprises an amino acid sequence having at least 80%, at least 85%, at least 90%, at least 95%, or at least 99% sequence identity to SEQ ID NO: 42.

45. The fusion protein of any one of claims 1 to 44 wherein the Fc region comprises an amino acid sequence comprising SEQ ID NO: 42.

46. The fusion protein of claim 1 wherein the Fc region comprises an amino acid sequence comprising SEQ ID NO: 42.

47. The fusion protein of any one of claims 1 to 46 further comprising a leader peptide wherein the leader peptide promotes expression and secretion in an expression system.

48. The fusion protein of claim 1 further comprising a leader peptide wherein the leader peptide promotes expression and secretion in an expression system.

49. The fusion protein of any one of claims 1 to 48 wherein the leader peptide has an amino acid sequence having at least 85%, at least 90%, at least 95%, or at least 99% sequence identity to SEQ ID NO: 44.

50. The fusion protein of any one of claims 1 to 49 wherein the leader peptide has an amino acid sequence comprising SEQ ID NOs: 44.

51. The fusion protein of any one of claims 1 to 50 wherein the fusion protein binds to a cell surface receptor having an affinity for a MHC-peptide complex.

52. The fusion protein of claim 1 wherein the fusion protein binds to a cell surface receptor having an affinity for a MHC-peptide complex.

53. The fusion protein of claim 51 or 52 wherein the cell surface receptors comprise B-cell receptors (BCRs) expressed on the surface of a hybridoma or B-cell.

54. The fusion protein of claim 53 wherein the fusion protein further binds antibodies secreted by the hybridoma or the B-cell.

55. The fusion protein of claim 51 or 52 wherein the cell surface receptors comprise T-cell receptors (TCRs).

56. A fusion protein comprising a β2-microglobulin, a flexible linker, a MHC class I heavy chain and a fragment crystallizable (Fc) region of an antibody.

57. The fusion protein of claim 56 wherein the flexible linker comprises at least 8, at least 10, at least 11, at least 12, at least 13, at least 14, at least 15, at least 16, at least 17, at least 18, at least 19 or at least 20 amino acid residues.

58. The fusion protein of claim 56 or 57 wherein the flexible linker comprises from 10 to 25 amino acid residues, or from 15 to 20 amino acid residues.

59. The fusion protein of any one of claims 56 to 58 wherein the flexible linker comprises 80% or more glycine, alanine and/or serine residues.

60. The fusion protein of any one of claims 56 to 59 wherein the flexible linker comprises an amino acid sequence comprising greater than 80%, greater than 85%, or greater than 90% sequence identity to SEQ ID NO: 35 or SEQ ID NO: 36.

61. The fusion protein of any one of claims 56 to 60 wherein the flexible linker comprises an amino acid sequence comprising SEQ ID NO: 35 or SEQ ID NO: 36.

62. The fusion protein of any one of claims 56 to 61 wherein β2 microglobulin comprises a mammalian β2 microglobulin.

63. The fusion protein of any one of claims 56 to 62 wherein the β2 microglobulin comprises a human or murine β2 microglobulin.

64. The fusion protein of any one of claims 56 to 63 wherein the β2 microglobulin comprises an amino acid sequence having greater than 80%, greater than 85%, greater than 90%, or greater than 95% sequence identity to SEQ ID NO: 23 or SEQ ID NO: 24.

65. The fusion protein of any one of claims 56 to 64 wherein the β2 microglobulin comprises an amino acid sequence comprising SEQ ID NO: 23 or SEQ ID NO: 24.

66. The fusion protein of any one of claims 56 to 65 wherein the MHC class I heavy chain comprises a human or a murine MHC class I heavy chain.

67. The fusion protein of any one of claims 56 to 66 wherein the MHC class I heavy chain comprises a human leukocyte antigen (HLA) heavy chain.

68. The fusion protein of any one of claims 56 to 67 wherein the MHC class I heavy chain comprises a HLA-A, a HLA-B, a HLA-C, a HLA-E, a HLA-F, or a HLA-G heavy chain.

69. The fusion protein of any one of claims 56 to 68 wherein the MHC class I heavy chain comprises a HLA-A, a HLA-B, or a HLA-C heavy chain.

70. The fusion protein of any one of claims 56 to 69 wherein the MHC class I heavy chain comprises HLA-A*02, HLA-A*11, or HLA-B*07.

71. The fusion protein of any one of claims 56 to 70 wherein the MHC class I heavy chain has an amino acid sequence having at least 80%, at least 85%, at least 90%, or at least 95% sequence identity to any one of SEQ ID NOs: 26, 28, and 30 and comprises at least one amino acid substitution selected from Y84A, Y84C, T80C, and A86C.

72. The fusion protein of any one of claims 56 to 71 wherein the MHC class I heavy chain comprises any one of SEQ ID NOs: 25-30.

73. The fusion protein of any one of claims 56 to 72 wherein the Fc region comprises the Fc region of a human, murine, rabbit, or goat antibody.

74. The fusion protein of any one of claims 56 to 73 wherein the Fc region comprises the Fc region of a human or murine antibody.

75. The fusion protein of any one of claims 56 to 74 wherein the Fc region comprises an Fc segment of an IgG antibody.

76. The fusion protein of any one of claims 56 to 75 wherein the Fc region is capable of initiating complement.

77. The fusion protein of any one of claims 56 to 76 wherein the Fc region comprises an amino acid sequence having at least 80%, at least 85%, at least 90%, at least 95%, or at least 99% sequence identity to SEQ ID NO: 42.

78. The fusion protein of any one of claims 56 to 77 wherein the Fc region comprises an amino acid sequence comprising SEQ ID NO: 42.

79. A dimer comprising two fusion proteins of any one of claims 1 to 78 wherein the Fc regions of the fusion proteins are covalently linked.

80. The dimer of claim 79 wherein the Fc regions of the fusion proteins are covalently linked by one or more disulfide bonds.

81. A fusion protein complex comprising the fusion protein of any one of claims 56 to 78 and an antigen peptide, wherein the antigen peptide is complexed with the fusion protein.

82. The fusion protein complex of claim 81 wherein the antigen peptide comprises from 8 to 15, from 8 to 14, from 8 to 13, from 8 to 12, from 8 to 11, or from 8 to 10 amino acid residues.

83. The fusion protein complex of claim 81 or 82 wherein the antigen peptide comprises an antigen peptide that can bind to the MHC class I heavy chain.

84. The fusion protein complex of any one of claims 81 to 83 wherein the antigen peptide comprises a human leukocyte antigen-A (HLA-A) restricted peptide, a HLA-B restricted peptide, a HLA-C restricted peptide, a HLA-E restricted peptide, a HLA-F restricted peptide, or a HLA-G restricted peptide.

85. The fusion protein complex of any one of claims 81 to 84 wherein the antigen peptide comprises a HLA-A restricted peptide or a HLA-B restricted peptide.

86. The fusion protein complex of any one of claims 81 to 85 wherein the antigen peptide comprises a HLA-A*02 restricted peptide, a HLA-A*11 restricted peptide, or a HLA-B*07 restricted peptide.

87. The fusion protein complex of any one of claims 81 to 86 wherein the antigen peptide comprises an amino acid sequence having greater than 80%, greater than 85%, or greater than 90% sequence identity to any one of SEQ ID NOs: 1-22.

88. The fusion protein complex of any one of claims 81 to 87 wherein the antigen peptide comprises any one of SEQ ID NOs: 1-22.

89. The fusion protein complex of any one of claims 81 to 88 wherein the antigen peptide comprises any one of SEQ ID NOs: 1-4.

90. The fusion protein complex of claim 81 wherein the antigen peptide comprises any one of SEQ ID NOs: 1-4.

91. The fusion protein complex of any one of claims 81 to 90 wherein the antigen peptide stabilizes the fusion protein complex.

92. The fusion protein complex of any one of claims 81 to 91 wherein fusion protein complex binds to a cell surface receptor having an affinity for a MHC-peptide complex.

93. The fusion protein complex of claim 92 wherein the cell surface receptor comprises a B-cell receptor (BCR) expressed on the surface of a hybridoma or B-cell.

94. The fusion protein complex of claim 93 wherein the fusion protein complex further binds to antibodies secreted by the hybridoma or B-cell.

95. The fusion protein complex of claim 92 wherein the cell surface receptor comprises a T-cell receptor (TCR).

96. A nucleic acid comprising a nucleotide sequence encoding the fusion protein of any one of claims 1 to 78.

97. The nucleic acid of claim 96 wherein the nucleotide sequence comprises any one of SEQ ID NOs: 69-74.

98. An expression vector comprising the nucleic acid of claim 96 or 97.

99. A host cell comprising the expression vector of claim 98.

100. A pharmaceutical composition comprising the fusion protein of any one of claims 1 to 78 the dimer of claim 79 or 80, and/or the fusion protein complex of any one of claims 81 to 95.

101. A method of depleting a population of antigen specific cells expressing a surface receptor having an affinity for a MHC-peptide complex, the method comprising contacting the cells with an effective amount of the fusion protein of any one of claims 1 to 78 or the fusion protein complex of any one of claims 81 to 95.

102. The method of claim 101 wherein the cells are in vitro and the method further comprises contacting the cells with complement.

103. The method of claim 101 wherein the cells are located in a subject and the method further comprises administering a therapeutically effective amount of the fusion protein or fusion protein complex to the subject.

104. The method of any one of claims 101 to 103 wherein:
(a) the MHC Type I heavy chain of the fusion protein or fusion protein complex comprises the heavy chain of the MHC-peptide complex having an affinity for the surface receptor of the antigen specific cells; or
(b) the antigen peptide of the fusion protein or fusion protein complex comprises the peptide of the MHC-peptide complex having an affinity for the surface receptor of the antigen specific cells; or
(c) a combination of any thereof.

105. The method of any one of claim 101 or 104 wherein the cells are antigen specific B cells or antigen specific T cells.

106. The method of claim 105 wherein the cells are antigen specific B cells and the cell surface receptor comprises a B-cell receptor.

107. The method of claim 105 wherein the cells are antigen specific T-cells and the cell surface receptor comprises a T-cell receptor.

108. The method of claims 101 to 107 wherein the MHC-peptide complex comprises a HLA-peptide complex.

109. A method of treating antibody-mediated transplant rejection, in a subject in need thereof, wherein the antibody-mediated rejection is caused by antibodies having an affinity for a foreign HLA-peptide complex, the method comprising depleting a population of B cells that express a surface receptor having an affinity for the foreign HLA-peptide complex in the subject according to the method of any one of claims 101 to 108.

110. A method of treating organ transplant rejection, graft-versus-host disease, blood transfusion refractoriness in a subject in need thereof, the method comprising administering a therapeutically effective amount of the fusion protein of any one of claims 1 to 78 to the subject or the fusion protein complex of any one of claims 81 to 95.

111. The method of claim 110 wherein the subject in need thereof produces antibodies having an affinity for a foreign HLA-peptide complex and wherein administering the fusion protein or fusion protein complex depletes a population of B cells in the subject that express the antibodies.

112. The method of claim 110 wherein administering the fusion protein or fusion protein complex depletes a population of T-cells in the subject expressing a T-cell receptor (TCR) having affinity for the foreign HLA-peptide complex.

113. A method of treating antibody-mediated hemolysis in a subject in need thereof, the method comprising administering a therapeutically effective amount of the fusion protein of any one of claims 1 to 78 or the fusion protein complex of any one of claims 81 to 95 to the subject.

114. The method of any one of claims 110 to 113 wherein administering the fusion protein or fusion protein complex does not impair global humoral immunity in the subject.

115. The method of any one of claims 110 to 114 wherein the subject is a human or research animal.

116. The method of any one of claims 101 to 115 comprising administering a dimer of claim 79 or 80.

117. The method of any one of claims 101 to 116 comprising administering the pharmaceutical composition of claim 100.

118. An imaging agent comprising a fusion protein of any one of claims 1 to 78 conjugated to a signaling moiety.

119. The imaging agent of claim 118 wherein the signaling moiety comprises a fluorophore, a fluorochrome, a radioisotope, a positron emitting isotope, or any combination thereof.

120. A method of staining an antigen specific cell population, the method comprising contacting the antigen specific cells with an imaging agent of claim 118 or 119 and imaging the cells.

121. A single chain construct comprising a fusion protein (Fc), a peptide, a HLA, β2-microglobulin (β2m), and at least one flexible linker.

122. The single chain construct of claim 121, wherein the HLA is selected from HLA-A and HLA-B.

123. A method of antigen-specific depletion of B-cells in a subject comprising administering a therapeutically effective amount of the single chain construct of claim 121 or 122.

124. A method of treating allosensitization or antibody mediated rejection in transplant patients comprising administering a therapeutically effective amount of the single chain construct of claim 121 or 122.

125. A method of treating blood transfusion refractoriness or antibody-mediated hemolysis in a subject comprising administering a therapeutically effective amount of the single chain construct of claim 121 or 122.

126. A method of treating an antibody-mediated autoimmune disease in a subject comprising administering a therapeutically effective amount of the single chain construct of claim 121 or 222, wherein the single chain construct comprises an autoantigen.

127. The method of any one of claims 123 to 126 wherein the single chain construct spares global humoral immunity.


SEQUENCE LISTING (74 sequences)

SEQ ID NO: 1 - protein, 9 aa, Cytomegalovirus
NLVPMVATV

SEQ ID NO: 2 - protein, 9 aa, Vaccinia virus
LPCQLMYAL

SEQ ID NO: 3 - protein, 9 aa, Dengue virus
GPMKLVMAF

SEQ ID NO: 4 - protein, 9 aa, Dengue virus
HPGFTILAL

SEQ ID NO: 5 - protein, 9 aa, Ebstein-Barr Virus
GLCTLVAML

SEQ ID NO: 6 - protein, 9 aa, Influenza A virus
GILGFVFTL

SEQ ID NO: 7 - protein, 9 aa, Human melanoma
IMDQVPFSV

SEQ ID NO: 8 - protein, 9 aa, Human melanoma
YLEPGPVTV

SEQ ID NO: 9 - protein, 8 aa, Artificial Sequence
  Synthetic construct
SIINFEKL

SEQ ID NO: 10 - protein, 8 aa, Artificial Sequence
  Synthetic construct
SIINYEKL

SEQ ID NO: 11 - protein, 8 aa, Artificial Sequence
  Synthetic construct
SIYRYYGL

SEQ ID NO: 12 - protein, 8 aa, Artificial Sequence
  Synthetic construct
RGYVYQGL

SEQ ID NO: 13 - protein, 9 aa, Artificial Sequence
  Synthetic construct
QLSPFPFDL

SEQ ID NO: 14 - protein, 9 aa, Artificial Sequence
  Synthetic construct
YPHFMPTNL

SEQ ID NO: 15 - protein, 9 aa, Human leukemia
LLFGYPVYV

SEQ ID NO: 16 - protein, 9 aa, Influenza A virus
SRYWAIRTR

SEQ ID NO: 17 - protein, 10 aa, Human breast cancer
LIYDSSLCDL

SEQ ID NO: 18 - protein, 10 aa, Hepatitis B virus
FLPSDFFPSV

SEQ ID NO: 19 - protein, 9 aa, Hepatitis B virus
CLTFGRETV

SEQ ID NO: 20 - protein, 9 aa, Human Immunodeficiency Virus
SLYNTVATL

SEQ ID NO: 21 - protein, 10 aa, Human ovarian cancer
KLLGPHVEGL

SEQ ID NO: 22 - protein, 9 aa, Artificial Sequence
  Synthetic construct
VMAPRTLIL

SEQ ID NO: 23 - protein, 99 aa, Mus musculus [sequence omitted]

SEQ ID NO: 24 - protein, 99 aa, Homo sapiens [sequence omitted]

SEQ ID NO: 25 - protein, 281 aa, Artificial Sequence
  Synthetic construct
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPT

SEQ ID NO: 26 - protein, 281 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPT

SEQ ID NO: 27 - protein, 281 aa, Artificial Sequence
  Synthetic construct
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPT

SEQ ID NO: 28 - protein, 281 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPT

SEQ ID NO: 29 - protein, 281 aa, Artificial Sequence
  Synthetic construct
GSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQST

SEQ ID NO: 30 - protein, 281 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQST

SEQ ID NO: 31 - protein, 10 aa, Artificial Sequence
  Synthetic construct
GGGASGGGGS

SEQ ID NO: 32 - protein, 10 aa, Artificial Sequence
  Synthetic construct
GGGGSGGGGS

SEQ ID NO: 33 - protein, 15 aa, Artificial Sequence
  Synthetic construct
GGGASGGGGSGGGGS

SEQ ID NO: 34 - protein, 15 aa, Artificial Sequence
  Synthetic construct
GGGGSGGGGSGGGGS

SEQ ID NO: 35 - protein, 20 aa, Artificial Sequence
  Synthetic construct
GGGGSGGGGSGGGGSGGGGS

SEQ ID NO: 36 - protein, 20 aa, Artificial Sequence
  Synthetic construct
GGGSGGGSGGGSGGGSGGGS

SEQ ID NO: 37 - protein, 15 aa, Artificial Sequence
  Synthetic construct
CGGASGGGGSGGGGS

SEQ ID NO: 38 - protein, 15 aa, Artificial Sequence
  Synthetic construct
GCGASGGGGSGGGGS

SEQ ID NO: 39 - protein, 15 aa, Artificial Sequence
  Synthetic construct
GGCASGGGGSGGGGS

SEQ ID NO: 40 - protein, 15 aa, Artificial Sequence
  Synthetic construct
GGGCSGGGGSGGGGS

SEQ ID NO: 41 - protein, 15 aa, Artificial Sequence
  Synthetic construct
GGGACGGGGSGGGGS

SEQ ID NO: 42 - protein, 232 aa, Mus musculus
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 43 - protein, 232 aa, Artificial Sequence
  Synthetic construct
PRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 44 - protein, 20 aa, Artificial Sequence
  Synthetic construct
MARSVTLVFLVLVSLTGLYA

SEQ ID NO: 45 - protein, 678 aa, Artificial Sequence
  Synthetic construct
MARSVTLVFLVLVSLTGLYANLVPMVATVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 46 - protein, 678 aa, Artificial Sequence
  Synthetic construct
MARSVTLVFLVLVSLTGLYANLVPMVATVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTRSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 47 - protein, 678 aa, Artificial Sequence
  Synthetic construct
MARSVTLVFLVLVSLTGLYANLVPMVATVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 48 - protein, 678 aa, Artificial Sequence
  Synthetic construct
MARSVTLVFLVLVSLTGLYALPCQLMYALGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 49 - protein, 678 aa, Artificial Sequence
  Synthetic construct
MARSVTLVFLVLVSLTGLYAGPMKLVMAFGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 50 - protein, 678 aa, Artificial Sequence
  Synthetic construct
MARSVTLVFLVLVSLTGLYAHPGFTILALGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 51 - protein, 658 aa, Artificial Sequence
  Synthetic construct
NLVPMVATVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 52 - protein, 658 aa, Artificial Sequence
  Synthetic construct
NLVPMVATVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTRSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 53 - protein, 658 aa, Artificial Sequence
  Synthetic construct
NLVPMVATVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 54 - protein, 658 aa, Artificial Sequence
  Synthetic construct
LPCQLMYALGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 55 - protein, 658 aa, Artificial Sequence
  Synthetic construct
GPMKLVMAFGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 56 - protein, 658 aa, Artificial Sequence
  Synthetic construct
HPGFTILALGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 57 - DNA, 60 bp, Artificial Sequence
  Synthetic construct
atggctcgctcggtgaccctggtctttctggtgcttgtctcactgaccggtttgtatgct

SEQ ID NO: 58 - DNA, 27 bp, Cytomegalovirus
aacctggtgcccatggtggccaccgtg

SEQ ID NO: 59 - DNA, 27 bp, Vaccinia virus
ctgccctgccagctgatgtacgccctg

SEQ ID NO: 60 - DNA, 27 bp, Dengue virus
ggccccatgaagctggtgatggccttc

SEQ ID NO: 61 - DNA, 27 bp, Dengue virus
caccccggcttcaccatcctggccctg

SEQ ID NO: 62 - DNA, 45 bp, Artificial Sequence
  Synthetic construct
ggatgcggtgctagcggtggtggaggtagcggaggtggaggaagc

SEQ ID NO: 63 - DNA, 60 bp, Artificial Sequence
  Synthetic construct
ggcggtggtggttccggtggaggcggttccggaggtggtggatccggtggtggaggtagt

SEQ ID NO: 64 - DNA, 297 bp, Homo sapiens
atccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatg

SEQ ID NO: 65 - DNA, 843 bp, Artificial Sequence
  Synthetic construct
ggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctgctacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccacc

SEQ ID NO: 66 - DNA, 843 bp, Artificial Sequence
  Synthetic construct
ggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctgctacaaccagagcgaggacggttctcacaccatccagataatgtatggctgcgacgtggggccggacgggcgcttcctccgcgggtatcggcaggacgcctacgacggcaaggattacatcgccctgaacgaggacctgcgctcttggaccgcggcggacatggcagctcagatcaccaagcgcaagtgggaggcggcccatgcggcggagcagcagagagcctacctggagggccggtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccacc

SEQ ID NO: 67 - DNA, 843 bp, Artificial Sequence
  Synthetic construct
ggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatatacaaggcccaggcacagactgaccgagagagcctgcggaacctgcgcggctgctacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccacc

SEQ ID NO: 68 - DNA, 699 bp, Mus musculus
cccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 69 - DNA, 2037 bp, Artificial Sequence
  Synthetic construct
atggctcgctcggtgaccctggtctttctggtgcttgtctcactgaccggtttgtatgctaacctggtgcccatggtggccaccgtgggatgcggtgctagcggtggtggaggtagcggaggtggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggtggtggttccggtggaggcggttccggaggtggtggatccggtggtggaggtagtggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctgctacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccagatctcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 70 - DNA, 2037 bp, Artificial Sequence
  Synthetic construct
atggctcgctcggtgaccctggtctttctggtgcttgtctcactgaccggtttgtatgctaacctggtgcccatggtggccaccgtgggatgcggtgctagcggtggtggaggtagcggaggtggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggtggtggttccggtggaggcggttccggaggtggtggatccggtggtggaggtagtggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctgctacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccagatctcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcggtgcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 71 - DNA, 2037 bp, Artificial Sequence
  Synthetic construct
atggctcgctcggtgaccctggtctttctggtgcttgtctcactgaccggtttgtatgctaacctggtgcccatggtggccaccgtgggatgcggtgctagcggtggtggaggtagcggaggtggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggtggtggttccggtggaggcggttccggaggtggtggatccggtggtggaggtagtggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatatacaaggcccaggcacagactgaccgagagagcctgcggaacctgcgcggctgctacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccagatctcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 72 - DNA, 2037 bp, Artificial Sequence
  Synthetic construct
atggctcgctcggtgaccctggtctttctggtgcttgtctcactgaccggtttgtatgctctgccctgccagctgatgtacgccctgggatgcggtgctagcggtggtggaggtagcggaggtggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggtggtggttccggtggaggcggttccggaggtggtggatccggtggtggaggtagtggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatatacaaggcccaggcacagactgaccgagagagcctgcggaacctgcgcggctgctacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccagatctcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 73 - DNA, 2037 bp, Artificial Sequence
  Synthetic construct
atggctcgctcggtgaccctggtctttctggtgcttgtctcactgaccggtttgtatgctggccccatgaagctggtgatggccttcggatgcggtgctagcggtggtggaggtagcggaggtggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggtggtggttccggtggaggcggttccggaggtggtggatccggtggtggaggtagtggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatatacaaggcccaggcacagactgaccgagagagcctgcggaacctgcgcggctgctacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccagatctcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 74 - DNA, 2037 bp, Artificial Sequence
  Synthetic construct
atggctcgctcggtgaccctggtctttctggtgcttgtctcactgaccggtttgtatgctcaccccggcttcaccatcctggccctgggatgcggtgctagcggtggtggaggtagcggaggtggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggtggtggttccggtggaggcggttccggaggtggtggatccggtggtggaggtagtggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatatacaaggcccaggcacagactgaccgagagagcctgcggaacctgcgcggctgctacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccagatctcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga